(19) United States
(12) Patent Application Publication
(10) Pub. No.: US 2026/0141986 A1
(43) Pub. Date: May 21, 2026

(54) System and Method for Estimating Catalyst Trim Effectiveness in Mixed Metallocene Catalysts for Polymerization Processes

(71) Applicant: ExxonMobil Technology and Engineering Company, Spring, TX (US)

(72) Inventors: Liezhong Gong, Basking Ridge, NJ (US); Kevin A. Stevens, Somerville, NJ (US)

(21) Appl. No.: 19/144,031

(22) PCT Filed: Jan. 19, 2024
(86) PCT No.: PCT/US2024/012260

§ 371 Date: Jun. 27, 2025

Related U.S. Application Data
(60) Provisional application No. 63/482,719, filed on Feb. 1, 2023.

(30) Foreign Application Priority Data
Feb. 1, 2023 (US) 63482719

(51) Int. Cl. G16C 20/30; G16C 20/10; G16C 60/00

(52) U.S. Cl. CPC G16C 20/30; G16C 20/10; G16C 60/00

(57) ABSTRACT

An example method includes: (A) receiving a polymerization process dataset having properties of polyethylene (PE) resins prepared using a multi-component catalyst composition and different operating parameters of a gas phase reactor system; (B) setting a catalyst trim effectiveness value of the multi-component catalyst composition to an initial value; (C) training a respective process model for each of the properties of the PE resins of the polymerization process dataset using the catalyst trim effectiveness value to yield a set of trained process models; (D) optimizing the catalyst trim effectiveness value to determine a minimized sum of squared error (&SE) value of model residuals using the set of trained process models; (E) repeating steps (C) and (D) until a decrease in the minimized SSE value is less than a predefined threshold value; and (F) outputting the catalyst trim effectiveness value and the set of trained process models for the multi-component catalyst composition.

### CROSS REFERENCE TO RELATED APPLICATION

This application claims the benefit of U.S. Provisional Application 63/482,719 filed Feb. 1, 2023, entitled “SYSTEM AND METHOD FOR ESTIMATING CATALYST TRIM EFFECTIVENESS IN MIXED METALLOCENE CATALYSTS FOR POLYMERIZATION PROCESSES”, the entirety of which is incorporated by reference herein.

### FIELD

This disclosure generally relates to catalyst slurry mixtures. In particular, this disclosure relates to a soft sensor technique for characterizing catalyst slurry mixtures to estimate the effectiveness of catalyst trimming in polymerization processes, such as gas-phase polyethylene (GPPE) polymerization processes and slurry-phase polymerization processes.

### BACKGROUND

Supported multi-component catalysts are widely used in commercial scale polymer production as multi-component catalysts enable the production of multi-modal polymer resins. For example, supported dual-component catalysts include a support with two types of active sites disposed thereon. The relative contribution to polymerization between the first and second active sites determines the composition of the resulting multi-modal polymer resin, where the relative contribution of each active site is dependent upon the ratio of the active sites on the supported dual-component catalyst.

A method of producing a supported dual-component catalyst may include reacting a first catalyst component containing a precursor for the first type of active site and a second catalyst component containing a precursor for the second type of active site at conditions suitable to transform at least a portion of the precursor of the first and second types of active sites to the activated form. The mole ratio of the two types of active sites does not necessarily equal the mole ratio of the two precursors used in the preparation of the supported catalyst. There are likely several reasons for the disparity, including that the two precursors may have different activation energies, and sometimes referred to as activation efficiency, thereby leading to a disparate activation for the two catalyst precursors used during the catalyst preparation. Even if the mole ratio of the two catalyst precursors is kept constant during catalyst production, the mole ratio of the resulting two types of active sites on the supported catalyst may vary due to variability in relative activation energy in catalyst preparation.

Further, at present there is no analytical technique that can directly probe the ratio of the two types of active sites on the supported catalyst. Without knowledge of the ratio of the active sites in the dual-component catalyst system, it is generally not possible to determine the composition of the polymer resin product in advance of using the dual-component catalyst system. Ideally, the dual-component catalyst system would be characterized to determine the ratio of active sites in advance of using the dual-component catalyst system in a polymerization application such that the catalyst response in the reactor as well as the composition of the resin product can be more readily controlled.

References of potential interest in this regard include: U.S. Patent Publication No. US2020/0071437 and US2022/0033535, as well as U.S. Pat. Nos. 8,429,100; 6,546,379; 7,505,949; 6,243,696; 11,288,577; 11,203,653; 10,494,462; 9,963,528; and 10,865,259.

### SUMMARY

Disclosed herein is an example soft sensor system that includes at least one memory configured to store a polymerization process dataset for a multi-component catalyst composition, wherein the polymerization process dataset includes properties of polyethylene (PE) resins prepared using the multi-component catalyst composition and different operating parameters of a gas phase reactor system. The soft sensor system includes at least one processor configured to execute stored instruction to perform actions. The actions include: (A) setting a catalyst trim effectiveness value of the multi-component catalyst composition to an initial value; (B) training a respective process model for each of the properties of the PE resins of the polymerization process dataset using the catalyst trim effectiveness value of the multi-component catalyst composition to yield a set of trained process models; (C) optimizing the catalyst trim effectiveness value of the multi-component catalyst composition to determine a minimized sum of squared error (SSE) value of model residuals based on the set of trained process models; (D) repeating steps (B) and (C) until a decrease in the minimized SSE value is less than a predefined threshold value; and (E) outputting or storing the catalyst trim effectiveness value and the set of trained process models for the multi-component catalyst composition.

Further disclosed herein is an example method of operating a soft sensor system. The method includes: (A) receiving a polymerization process dataset for a multi-component catalyst composition, wherein the polymerization process dataset includes properties of polyethylene (PE) resins prepared using the multi-component catalyst composition and different operating parameters of a gas phase reactor system; (B) setting a catalyst trim effectiveness value of the multi-component catalyst composition to an initial value; (C) training a respective process model for each of the properties of the PE resins of the polymerization process dataset using the catalyst trim effectiveness value of the multi-component catalyst composition to yield a set of trained process models; (D) optimizing the catalyst trim effectiveness value of the multi-component catalyst composition to determine a minimized sum of squared error (SSE) value of model residuals based on the set of trained process models; (E) repeating steps (C) and (D) until a decrease in the minimized SSE value is less than a predefined threshold value; and (F) outputting or storing the catalyst trim effectiveness value and the set of trained process models for the multi-component catalyst composition.

Further disclosed herein is an example non-transitory, computer-readable medium storing instructions executable by a processor of a soft sensor system. The instructions include instructions to: (A) receive a polymerization process dataset for a multi-component catalyst composition, wherein the polymerization process dataset includes properties of polyethylene (PE) resins prepared using the multi-component catalyst composition and different operating parameters of a gas phase reactor system; (B) set a catalyst trim effectiveness value of the multi-component catalyst composition to an initial value; (C) train a respective process model for each of the properties of the PE resins of the polymerization process dataset using the catalyst trim effectiveness value of the multi-component catalyst composition to yield a set of trained process models; (D) optimize the catalyst trim effectiveness value of the multi-component catalyst composition to determine a minimized sum of squared error (SSE) value of model residuals based on the set of trained process models; (E) repeat steps (C) and (D) until a decrease in the minimized SSE value is less than a predefined threshold value; and (F) output or store the catalyst trim effectiveness value and the set of trained process models for the multi-component catalyst composition.

These and other features and attributes of the disclosed methods and systems of the present disclosure and their advantageous applications and/or uses will be apparent from the detailed description that follows.

### BRIEF DESCRIPTION OF THE DRAWINGS

To assist those of ordinary skill in the relevant art in making and using the subject matter hereof, reference is made to the appended drawings, wherein:

FIG. 1 is a schematic of a gas-phase reactor system that includes a soft sensor system, in accordance with certain embodiments of the present disclosure.

FIG. 2A illustrates an example method of producing a dual-component catalyst system and using the dual-component catalyst system in a production of a multi-modal polymer, in accordance with certain embodiments of the present disclosure.

FIG. 2B illustrates the contribution of active sites of a dual-component catalyst, in accordance with certain embodiments of the present disclosure.

FIG. 2C illustrates the preparation of a dual-component catalyst system and the ratio of the two types of activated sites on the dual-component catalyst, in accordance with certain embodiments of the present disclosure.

FIG. 3A illustrates a production scheme for a post-trim catalyst, in accordance with certain embodiments of the present disclosure.

FIG. 3B illustrates the relative ratio of the two types of active sites in a post-trim catalyst, in accordance with certain embodiments of the present disclosure.

FIG. 4 is a flow diagram illustrating a process of generating a gas-phase polyethylene (GPPE) polymerization process dataset, in accordance with certain embodiments of the present disclosure.

FIG. 5 is a flow diagram illustrating a process of determining a catalyst trim effectiveness value (η) and trained process models for a multi-component catalyst composition, in accordance with certain embodiments of the present disclosure.

FIG. 6 is a graph illustrating an example plot of minimized sum of squared error (SSE) values of model residuals versus the number of iterations of optimization of the catalyst trim effectiveness value (η), in accordance with certain embodiments of the present disclosure.

FIG. 7 is a flow diagram illustrating a process of operating the soft sensor system using the catalyst trim effectiveness value (η) and the trained process models to predict properties of a polyethylene (PE) resin to be produced using a multi-component catalyst composition, in accordance with certain embodiments of the present disclosure.

FIG. 8 is a flow diagram illustrating a process of operating the smart sensor system using the catalyst trim effectiveness value (η), the trained process models, and desired properties of a PE resin to be produced to determine predicted operating parameters for the GPPE polymerization process, in accordance with certain embodiments of the present disclosure.

### DETAILED DESCRIPTION

Disclosed herein are methods of estimating trim effectiveness of supported catalyst in slurry, as well as methods of predicting the material properties of olefin products based on the estimated trim effectiveness. Gas-phase polymerization in a fluidized bed is an industrial process used in polymerizing ethylene and ethylene comonomers to produce polyethylene polymer and copolymer compositions. It is generally known in the art that a polyolefin's composition distribution (CD) and molecular weight distribution (MWD) affect attributes of the polyolefin. To reduce or to avoid certain trade-offs among desirable attributes, bimodal polymers have become increasingly important in the polyolefins industry. Catalyst design and support technology have allowed for the development of single-reactor bimetallic (dual component) catalyst systems capable of producing bimodal polyethylene. The active sites' composition (i.e., the mole ratio of the two types of active sites) in dual-component catalyst systems often differs from the mole ratio of the two catalyst compound precursors used in making the dual-component catalyst. The evaluation of the active sites' composition of a dual-component catalyst system has relied on polymerization testing, as no established or commercial analytical methods offer a direct assessment of the active sites ratio of the catalyst.

A supported catalyst is added to a diluent to form a catalyst slurry and pumped to a polymerization reactor. A catalyst solution can be added (i.e., “trimmed”) to the catalyst slurry to adjust one or more properties “in-situ” of polymer being formed in a reactor. However, such “trim” processes are limited because the post-trim catalyst is typically not characterized before introduction into the polymerization reactor, as described above.

As used herein, the indefinite article “a” or “an” shall mean “at least one” unless specified to the contrary or the context clearly indicates otherwise. Thus, embodiments using “an alpha-olefin” include embodiments where one, two or more alpha-olefins are used, unless specified to the contrary or the context clearly indicates that only one alpha-olefin is used.

As used herein, “wt. %” means percentage by weight, “vol %” means percentage by volume, “mol %” means percentage by mole. “ppm” means parts per million, and “ppm wt” and “wppm” are used interchangeably and mean parts per million on a weight basis. All concentrations herein, unless otherwise stated, are expressed on the basis of the total amount of the composition in question. Unless otherwise stated, temperatures are provided in degrees Celsius (° C.).

An “olefin” is a linear, branched, or cyclic compound of carbon and hydrogen having at least one double bond. For purposes of this specification and the claims appended thereto, when a polymer or copolymer is referred to as including an olefin, e.g., ethylene and at least one C₃ to C₂₀ α-olefin, the olefin present in such polymer or copolymer is the polymerized form of the olefin. For example, when a copolymer is said to have an “ethylene” content of 35 wt. % to 55 wt %, it is understood that the repeating unit/mer unit or simply unit in the copolymer is derived from ethylene in the polymerization reaction, and the derived units are present at 35 wt. % to 55 wt. %, based on a weight of the copolymer. For the purposes of the present disclosure, ethylene shall be considered an α-olefin.

A “polymer” has two or more of the same or different repeating units/mer units or simply units. A “homopolymer” is a polymer having units that are the same. A “copolymer” is a polymer having two or more units that are different from each other. A “terpolymer” is a polymer having three units that are different from each other. The term “different” as used to refer to units indicates that the units differ from each other by at least one atom or are different isomerically. The definition of copolymer, as used herein, includes terpolymers and the like. Likewise, the definition of polymer, as used herein, includes homopolymers, copolymers, and the like. Furthermore, the terms “polyethylene copolymer”, “ethylene copolymer”, and “ethylene-based polymer” are used interchangeably to refer to a copolymer that includes at least 50 mol % of units derived from ethylene.

Nomenclature of elements and groups thereof used herein are pursuant to the NEW NOTATION published in HAWLEYS CONDENSED CHEMICAL DICTIONARY, Thirteenth Edition, John Wiley & Sons, Inc., (1997) (reproduced there with permission from IUPAC), unless reference is made to the Previous IUPAC form noted with Roman numerals (also appearing in the same), or unless otherwise noted. IUPAC refers to the International Union of Pure and Applied Chemistry.

As used herein, the term “slurry catalyst mixture” refers to a contact product that includes at least one catalyst compound and a carrier fluid (e.g., mineral oil), and optionally one or more of an activator, a co-activator, and a support. In a preferred embodiment, the slurry catalyst mixture includes a contact product that includes at least two catalyst compounds and the carrier fluid (e.g., mineral oil), and optionally one or more of an activator, a co-activator, and a support.

As used herein, the term “catalyst system” refers to a combination of at least one catalyst compound, an optional activator, an optional co-activator, and an optional support material. As such, in some embodiments the catalyst system can include only a single catalyst compound when the optional activator, the optional co-activator, and the optional support material are not present. In other embodiments, the catalyst system can include only two or more catalyst compounds when the optional activator, the optional co-activator, and the optional support material are not present. For the purposes of the present disclosure, when, catalyst systems are described as including neutral stable forms of the components, as it is well understood by one of ordinary skill in the art, the ionic form of the component is the form that reacts with the monomers to produce polymers. Catalyst systems, catalysts, and activators of the present disclosure are intended to embrace ionic forms in addition to the neutral forms of the compounds/components.

A metallocene catalyst is an organometallic compound with at least one π-bound cyclopentadienyl moiety (or substituted cyclopentadienyl moiety) and more frequently two π-bound cyclopentadienyl moieties or substituted cyclopentadienyl moieties bonded to a transition metal. In the description herein, the metallocene catalyst may be described as a catalyst precursor, a pre-catalyst compound, metallocene catalyst compound or a transition metal compound, and these terms are used interchangeably. An “anionic ligand” is a negatively charged ligand that donates one or more pairs of electrons to a metal ion. For purposes of the present disclosure, in relation to metallocene catalyst compounds, the term “substituted” means that a hydrogen group has been replaced with a hydrocarbyl group, a heteroatom, or a heteroatom containing group. For example, methyl cyclopentadiene (Cp) is a Cp group substituted with a methyl group.

“Alkoxides” include an oxygen atom bonded to an alkyl group that is a C₁ to C₁₀ hydrocarbyl. The alkyl group may be straight chain, branched, or cyclic. The alkyl group may be saturated or unsaturated. In at least one embodiment, the alkyl group may comprise at least one aromatic group.

“Asymmetric” as used in connection with the instant indenyl compounds means that the substitutions at the 4 positions are different, or the substitutions at the 2 positions are different, or the substitutions at the 4 positions are different and the substitutions at the 2 positions are different.

The properties and performance of polyethylene compositions can be advanced by the combination of: (1) varying one or more reactor conditions such as reactor temperature, hydrogen concentration, comonomer concentration, and so on; and (2) selecting and feeding a dual catalyst system having a first catalyst and second catalyst; the dual catalyst system may advantageously be trimmed as needed with additional first and/or second catalyst. Such catalyst trim processes provide for ready adjustment of polyethylene properties by permitting adjustment of ratios of first and second catalyst in the dual catalyst system fed to the reactor.

In various embodiments in accordance with the present disclosure, the slurry catalyst mixture can include a first catalyst compound that can be a “high molecular weight component” and a second catalyst compound that can be a “low molecular weight component.” In other words, the first catalyst can provide primarily for a high molecular-weight portion of the polymer and the second catalyst can provide primarily for a low molecular weight portion of the polymer (e.g., the first catalyst tends to produce relatively higher-molecular-weight polymer chains; while the second catalyst tends to produce relatively lower-molecular-weight polymer chains). In at least one embodiment, a dual catalyst system can be present in a catalyst pot of a reactor system, and a molar ratio of the first catalyst compound to the second catalyst compound of the dual catalyst system can be from 99:1 to 1:99, such as from 90:10 to 10:90, such as from 85:15 to 50:50, such as from 75:25 to 50:50, such as from 60:40 to 40:60. Consistent with the above note regarding trim catalyst systems, the first catalyst compound and/or the second catalyst compound can be added to a polymerization process as a trim catalyst to adjust the molar ratio of the first catalyst compound to the second catalyst compound. In at least one embodiment, the first catalyst compound and the second catalyst compound are each a metallocene catalyst compound.

### Gas Phase Reactor

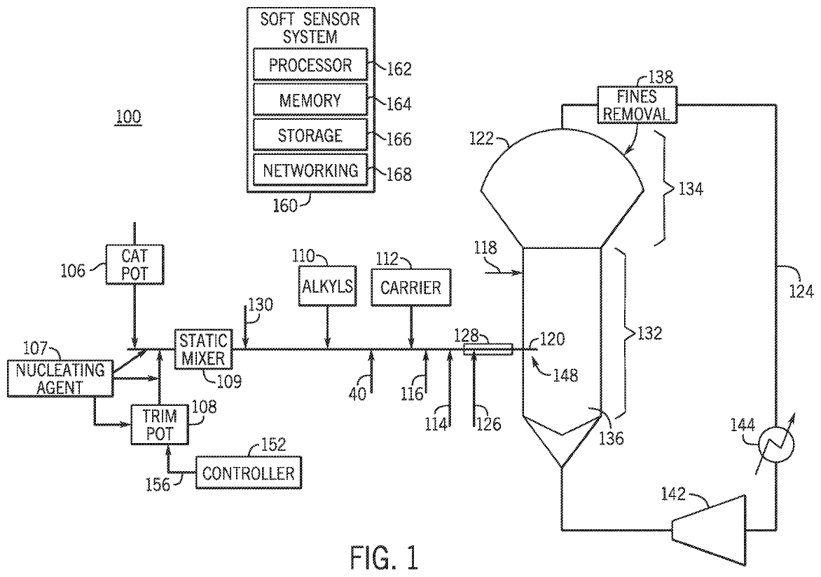

FIG. 1 is a schematic of a gas-phase reactor system 100, showing the addition of at least two catalysts, at least one of which is added as a trim catalyst. The catalyst slurry mixture from catalyst pot 106 and solution catalyst mixture from trim pot 108 can be mixed in-line. For example, the solution catalyst mixture and catalyst slurry mixture can be mixed by utilizing a mixer, such as static mixer 109 and/or an agitating vessel. Alternatively, any suitable mixer may be used including mixing in a conduit, mixing using a mixing apparatus, or mixing in a continuously agitated tank, for example. Any mixer capable of contacting the solution catalyst mixture and catalyst slurry mixture can be used.

Catalyst pot 106 contains a first catalyst slurry mixture. The first catalyst slurry mixture can be prepared by any suitable method, including, for example, by mixing particles of the first catalyst with mineral oil. The catalyst pot 106 can be an agitated holding tank configured to keep the solids concentration homogenous. In at least one embodiment, the catalyst pot 106 can be maintained at an elevated temperature (relative to room temperature), such as from 30° C. to 80° C. Alternatively, the catalyst pot 106 can be maintained from 30° C. to 40° C., from 40° C. to 50° C. from 50° C. to 60° C., from 60° C. to 80° C., or any ranges therebetween. Elevated temperatures can be obtained by electrically heat tracing the catalyst pot 106 using, for example, a heating blanket. Maintaining the catalyst pot 106 at an elevated temperature can further reduce or eliminate solid residue formation on vessel walls, which could otherwise slide off of the walls and cause plugging in downstream delivery lines. In at least one embodiment, catalyst pot 106 can have a volume from 0.5 cubic meters (m³) to 8.0 m³. Alternatively, the volume of the catalyst pot 106 may range from 0.5 m³ to 1.0 m³, 1.0 m³ to 2.0 m³, 2.0 m³ to 3.0 m³, 3.0 m³ to 4.0 m³, 4.0 m³ to 5.0 m³, 5.0 m³ to 6.0 m³, 6.0 m³ to 7.0 m³, 7.0 m³ to 8.0 m³, or any ranges therebetween.

The catalyst pot 106 can be maintained at pressure of 1.0 bar to 4.0 bar. Alternatively, the pressure of the catalyst pot 106 may range from 1.0 bar to 1.5 bar, 1.5 bar to 2.0 bar, 2.0 bar to 2.5 bar, 2.5 bar to 3.0 bar, 3.0 bar to 3.5 bar, 3.5 bar to 4.0 bar, or any ranges therebetween. In at least one embodiment, piping 130 and piping 140 of the gas-phase reactor system 100 can be maintained at an elevated temperature (relative to room temperature), such as from 30° C. to 80° C. Alternatively, the temperature of the piping 130 and 140 may range from 30° C. to 40° C., from 40° C. to 50° C., from 50° C. to 60° C., from 60° C. to 80° C., or any ranges therebetween. Elevated temperatures can be obtained by electrically heat tracing the piping 130 and/or the piping 140 using, for example, a heating blanket. Maintaining the piping 130 and/or the piping 140 at an elevated temperature can provide the same or similar benefits as described for an elevated temperature of catalyst pot 106.

A solution catalyst mixture, prepared by mixing a solvent and at least one second catalyst and/or activator, can be placed in another vessel, such as a trim pot 108. Trim pot 108 can have a volume of 0.5 m³ to 1.0 m³, 1.0 m³ to 2.0 m³, 2.0 m³ to 3.0 m³, 3.0 m³ to 4.0 m³, 4.0 m³ to 5.0 m³, 5.0 m³ to 6.0 m³, 6.0 m³ to 7.0 m³, 7.0 m³ to 8.0 m³, or any ranges therebetween. The trim pot 108 can be maintained at an elevated temperature (relative to room temperature), such as from 30° C. to 80° C. Alternatively, the temperature of the trim pot 108 may range from 30° C. to 40° C. from 40° C. to 50° C. from 50° C. to 60° C. from 60° C. to 80° C. or any ranges therebetween. The trim pot 108 can be heated by electrically heat tracing the trim pot 108, for example, via a heating blanket. Maintaining the trim pot 108 at an elevated temperature can provide reduced or eliminated foaming in piping 130 and or piping 140 when the catalyst slurry mixture from catalyst pot 106 is combined in-line (also referred to herein as “on-line”) with the solution catalyst mixture from trim pot 108.

The catalyst slurry mixture can then be combined in-line with the solution catalyst mixture to form a slurry/solution catalyst mixture or final catalyst composition. Optionally, a nucleating agent 107, such as silica, alumina, fumed silica or any other suitable particulate matter can be added to the slurry, and/or the solution in-line, and/or in catalyst pot 106 or trim pot 108 (this optional addition is illustrated in the example of FIG. 1). Similarly, additional activators or catalyst compounds can be added in-line. For example, a second catalyst slurry mixture that includes a different catalyst can be introduced from a second catalyst pot or “cat pot” (which may include wax and mineral oil). The two catalyst slurry mixtures can be used, as the catalyst system with or without the addition of a solution catalyst mixture from the trim pot 108. Controller 152 may include a computer system, microcontrollers, programmable logic controller, or any other control system capable of monitoring and adjusting process variables within gas-phase reactor system 100. Controller 152 also may include other devices to carry out the control, including sensors, actuators, and a control algorithm.

The catalyst slurry mixture and solution catalyst mixture can be mixed in-line. For example, the solution catalyst mixture and catalyst slurry mixture can be mixed by utilizing a mixer such as static mixer 109 and/or an agitating vessel. In some embodiments, the mixer may include static mixer 109 followed by an agitating vessel. The mixing of the catalyst slurry mixture and the solution catalyst mixture is sufficient to allow the catalyst compound in the solution catalyst mixture to disperse in the catalyst slurry mixture, such that the catalyst component (originally in the solution) migrates to the supported activator (originally present in the slurry). The combination can form a uniform dispersion of catalyst compounds on the supported activator forming the catalyst composition. The length of time that the slurry and the solution can be contacted can be in a range of 1 minute to 4 hours. Alternatively, this length of time may range from 1 minute to 10 minutes, 10 minutes to 30 minutes, 30 minutes to 1 hour, 1 hour to 1.5 hours, 1.5 hours to 2 hours, 2 hours to 2.5 hours, 2.5 hours to 3 hours, 3 hours to 3.5 hours, 3.5 hours to 4 hours, or any ranges therebetween.

Static mixer 109 of the gas-phase reactor system 100 can be maintained at an elevated temperature (relative to room temperature), such as from 30° C. to 80° C. Alternatively, the temperature of the static mixer 109 may range from 30° C. to 40° C., from 40° C. to 50° C., from 50° C. to 60° C. from 60° C. to 80° C., or any ranges therebetween. The elevated temperature of the static mixer 109 can be obtained by electrically heat tracing the static mixer 109 using, for example, a heating blanket. Maintaining static mixer 109 at an elevated temperature can provide reduced or eliminated foaming in static mixer 109 and can promote mixing of the catalyst slurry mixture and catalyst solution (as compared to lower temperatures), which reduces run times in the static mixer and for the overall polymerization process.

Optionally, an aluminum alkyl, an ethoxylated aluminum alkyl, an aluminoxane, an anti-static agent or a borate activator, such as a C₁ to C₁₅ alkyl aluminum (for example tri-isobutyl aluminum, trimethyl aluminum or the like), a C₁ to C₁₅ ethoxylated alkyl aluminum or methyl aluminoxane, ethyl aluminoxane, isobutylaluminoxane, modified aluminoxane or the like can be added to the mixture of the slurry/solution catalyst mixture in line. The alkyls, antistatic agents, borate activators and/or aluminoxanes can be added from an alkyl vessel 110 directly to the combination of the solution catalyst mixture and the catalyst slurry mixture, or, can be added via an additional alkane (e.g., hexane, heptane, and or octane) carrier stream, for example, from a carrier vessel 112. The additional alkyls, antistatic agents, borate activators and/or aluminoxanes may be present at up to 500 ppm, at 1 to 300 ppm, at 10 ppm to 300 ppm, or at 10 to 100 ppm. A carrier gas 114 such as nitrogen, argon, ethane, propane, and the like, can be added in-line to the mixture of the slurry and the solution. In embodiments, the carrier gas can be added at the rate of 0.5 kilograms per hour (kg/hr) to 45 kg/hr. Alternatively, the carrier gas addition rate may range from 0.5 kg/hr to 1.0 kg/hr, 1.0 kg/hr to 10 kg/hr, 10 kg/hr to 20 kg/hr, 20 kg/hr to 30 kg/hr, 30 kg/hr to 45 kg/hr, or any ranges therebetween.

A liquid carrier stream can be introduced into the combination of the solution catalyst mixture and the catalyst slurry mixture. The mixture of the solution, the slurry, and the liquid carrier stream can pass through a mixer or length of tube for mixing before being contacted with a gaseous carrier stream. Similarly, a comonomer 116, such as hexene, another alpha-olefin, or diolefin, may be added in-line to the mixture of the slurry and the solution.

A gas stream 126, such as cycle gas, or recycle gas 124, monomer, nitrogen, or other materials can be introduced into an injection nozzle 148, which can include a support tube 128 at least partially surrounded by an injection tube 120. The slurry/solution catalyst mixture can be passed through the injection tube 120 into fluidized bed reactor 122. In at least one embodiment, the injection tube 120 may aerosolize the slurry/solution mixture. Any number of suitable tubing sizes and configurations may be used to aerosolize and/or inject the slurry/solution mixture.

Fluidized bed reactor 122 can include a reaction zone 132 and a velocity reduction zone 134. The reaction zone 132 can include a bed 136 that can include growing polymer particles, formed polymer particles, a minor amount of catalyst particles fluidized by the continuous flow of the gaseous monomer, and diluent to remove the heat of polymerization through the reaction zone. Optionally, a portion of the recycle gas 124 can be cooled and compressed to form liquids that can increase the heat removal capacity of the circulating gas stream when readmitted to the reaction zone. A suitable rate of gas flow can be readily determined by experimentation. Make-up of gaseous monomer to the circulating gas stream can be at a rate equal to the rate at which particulate polymer product and monomer associated therewith is withdrawn from the reactor, and the composition of the gas passing through the reactor can be adjusted to maintain an essentially steady state gaseous composition within the reaction zone. The gas leaving the reaction zone 132 can be passed to the velocity reduction cone 134 where entrained particles can be removed, for example, via gravity separation as the entrained particles slow and fall back to the reaction zone 132. If desired, finer entrained particles and dust can be removed in a separation system 138, such as a cyclone separator and/or fines filter. The recycle gas 124 can be passed through a heat exchanger 144 where at least a portion of the heat of polymerization can be removed. The gas can then be compressed in a compressor 142 and returned to the reaction zone 132. To promote formation of particles in fluidized bed reactor 122, a nucleating agent 118 (e.g., fumed silica) can be added directly into fluidized bed reactor 122. Conventional trim polymerization processes include introducing a nucleating agent into the polymerization reactor. Furthermore, when a metallocene catalyst or other similar catalyst is used in the gas phase reactor, oxygen or fluorobenzene can be added to the fluidized bed reactor 122 directly or to the gas stream 126 to control the polymerization rate.

FIG. 1 is not limiting, as additional solution catalyst mixtures and/or catalyst slurry mixtures can be used. For example, a catalyst slurry mixture can be combined with two or more solution catalyst mixtures having the same or different catalyst compounds and or activators. Likewise, the solution catalyst mixture can be combined with two or more catalyst slurry mixtures, each having the same or different supports, and the same or different catalyst compounds and/or activators. Similarly, two or more catalyst slurry mixtures can be combined with two or more solution catalyst mixtures, for example in-line, where the catalyst slurry mixtures each include the same or different supports and can include the same or different catalyst compounds and or activators and the solution catalyst mixtures can include the same or different catalyst compounds and/or activators. For example, the catalyst slurry mixture can contain a supported activator and two different catalyst compounds, and two solution catalyst mixtures, each containing one of the catalysts in the slurry, wherein each can be independently combined, in-line, with the slurry.

The reactor temperature of the fluid bed process can be in a range of 30° C. to 200° C. Alternatively, the reactor temperature may range from 30° C. to 40° C., 40° C. to 50° C., 50° C. to 80° C., 80° C. to 100° C. 100° C. to 150° C. 150° C. to 200° C., or any ranges therebetween. In general, the reactor can be operated at a suitable temperature that accounts for the sintering temperature of the polymer product within the reactor. Thus, the upper temperature limit in various embodiments can be the melting temperature of the polyethylene copolymer produced in the reactor. However, higher temperatures can result in narrower molecular weight distributions that can be improved by the addition of a catalyst, or other co-catalysts.

Hydrogen gas can be used in the polymerization process to help control or otherwise adjust the final properties of the polyolefin. Using certain catalyst systems, increasing concentrations (partial pressures) of hydrogen can increase a flow index such as the melt index of the polyethylene polymer. The melt index can thus be influenced by the hydrogen concentration. The amount of hydrogen in the polymerization can be expressed as a mole ratio relative to the total polymerizable monomer, for example, ethylene, or a blend of ethylene and hexene or propylene. The amount of hydrogen used in the polymerization process can be an amount that achieves the desired melt index of the final polyolefin polymer. For example, the mole ratio of hydrogen to total monomer (H₂:monomer) can be 0.0001 or greater, 0.0005 or greater, or 0.001 or greater. Further, the mole ratio of hydrogen to total monomer (H₂:monomer) can be 10 or less, 5 or less, 3 or less, or 0.10 or less. A range for the mole ratio of hydrogen to monomer can include any combination of any upper mole ratio limit with any lower mole ratio limit described herein. In various embodiments, the amount of hydrogen in the reactor at any time can range to up to 5,000 ppm, up to 4,000 ppm, up to 3,000 ppm, or from 50 ppm to 5.000 ppm, or from 50 ppm to 2.000 ppm. The amount of hydrogen in the reactor can range from 1 ppm, 50 ppm, or 100 ppm to 400 ppm, 800 ppm, 1,000 ppm, 1.500 ppm, or 2,000 ppm, based on weight. Further, the ratio of hydrogen to total monomer (H₂:monomer) can be 0.00001:1 to 2:1, 0.005:1 to 1.5:1, or 0.0001:1 to 1:1. The one or more reactor pressures in a gas phase process (either single stage or two or more stages) can vary from 690 kilopascal (kPa) to 1,379 kPa. or from 1,724 kPa to 2,414 kPa or from 2.759 kPa to 3.448 kPa.

The gas phase reactor can be capable of producing from 10 kilograms per hour (kg/hr) to greater than 455 kg/hr, greater than 4,540 kg/hr, greater than 11.300 kg/hr. greater than 15,900 kg/hr. greater than 22.700 kg/hr. or greater than 29.000 kg/hr to 45.500 kg/hr of polymer.

In embodiments, the polymer product can have a melt index ratio (MIR) ranging from 10 to less than 300, or, in many embodiments, from 20 to 66, such as 25 to 55. The melt index (MI, I2) can be measured in accordance with ASTM D-1238-20, wherein the melt index is determined using a 2.1 kg loading, and the melt index ratio is determined from the ratio of a 21.6 kg loading to the 2.1 kg loading, both at 190° C.

The polymer product can have a density ranging from 0.89 grams per cubic centimeter (g/cm³), 0.90 g/cm³, 0.91 g/cm³, or 0.92 g/cm³ to 0.93 g/cm³, 0.95 g/cm³, 0.96 g/cm³, or 0.97 g/cm³. Density of the polymer product can be determined in accordance with ASTM D-792-20. The polymer can have a bulk density, measured in accordance with ASTM D-1895-17 method B, of from 0.25 g/cm³ to 0.5 g/cm³. For example, the bulk density of the polymer can be from 0.30 g/cm³, 0.32 g/cm³, or 0.33 g/cm³ to 0.40 g/cm³, 0.44 g/cm³, or 0.48 g/cm³.

The polymerization process according to various embodiments can include contacting one or more olefin monomers with a catalyst slurry mixture that can include mineral oil and catalyst particles. The one or more olefin monomers can be ethylene and/or propylene and the polymerization process can include heating the one or more olefin monomers and the catalyst system to 70° C. or more to form ethylene polymers or propylene polymers.

Monomers useful herein include substituted or unsubstituted C₂ to C₄₀ alpha-olefins, such as C₂ to C₂₀ alpha-olefins, such as C₂ to C₁₂ alpha-olefins, such as ethylene, propylene, butene, pentene, hexene, heptene, octene, nonene, decene, undecene, dodecene, and isomers thereof. For example, the monomer can include ethylene and one or more optional comonomers selected from propylene or C₄ to C₄₀ olefins, such as C₄ to C₂₀ olefins, such as C₆ to C₁₂ olefins. The C₄ to C₄₀ olefin monomers may be linear, branched, or cyclic. The C₄ to C₄₀ cyclic olefins may be strained or unstrained, monocyclic or polycyclic, and may optionally include heteroatoms and/or one or more functional groups.

In some embodiments, the C₂ to C₄₀ alpha-olefin monomers and optional comonomers include ethylene, propylene, butene, pentene, hexene, heptene, octene, nonene, decene, undecene, dodecene, norbornene, norbornadiene, dicyclopentadiene, cyclopentene, cycloheptene, cyclooctene, cyclooctadiene, cyclododecene, 7-oxanorbornene, 7-oxanorbornadiene, substituted derivatives thereof, and isomers thereof, such as hexene, heptene, octene, nonene, decene, dodecene, cyclooctene, 1,5-cyclooctadiene, 1-hydroxy-4-cyclooctene, 1-acetoxy-4-cyclooctene, 5-methylcyclopentene, cyclopentene, dicyclopentadiene, norbornene, norbornadiene, and their respective homologs and derivatives, such as norbornene, norbornadiene, and dicyclopentadiene.

In various embodiments, one or more dienes can be present in the polymer product at up to 10 wt. %, such as at 0.00001 to 1.0 wt. %, such as 0.002 to 0.5 wt. %, such as 0.003 to 0.2 wt. %, based upon the total weight of the composition, 500 ppm or less of diene can be added to the polymerization, such as 400 ppm or less, or such as 300 ppm or less; also or instead, at least 50 ppm of diene can be added to the polymerization, or 100 ppm or more, or 150 ppm or more.

Diene monomers include any hydrocarbon structure, such as C₄ to C₃₀, having at least two unsaturated bonds, where at least two of the unsaturated bonds are readily incorporated into a polymer by either a stereospecific or a non-stereospecific catalyst(s). The diene monomers can be selected from alpha, omega-diene monomers (i.e. di-vinyl monomers). The diolefin monomers are linear di-vinyl monomers, such as those containing from 4 to 30 carbon atoms. Examples of dienes can include, but are not limited to, butadiene, pentadiene, hexadiene, heptadiene, octadiene, nonadiene, decadiene, undecadiene, dodecadiene, tridecadiene, tetradecadiene, pentadecadiene, hexadecadiene, heptadecadiene, octadecadiene, nonadecadiene, icosadiene, heneicosadiene, docosadiene, tricosadiene, tetracosadiene, pentacosadiene, hexacosadiene, heptacosadiene, octacosadiene, nonacosadiene, triacontadiene, 1,6-heptadiene, 1,7-octadiene, 1,8-nonadiene, 1,9-decadiene, 1,10-undecadiene, 1,11-dodecadiene, 1,12-tridecadiene, 1,13-tetradecadiene, and low molecular weight polybutadienes (e.g., weight-averaged molecular weight (Mw) less than 1000 grams per mol (g/mol)). Cyclic dienes include cyclopentadiene, vinylnorbomene, norbomadiene, ethylidene norbomene, divinylbenzene, dicyclopentadiene or higher ring containing diolefins with or without substituents at various ring positions.

The catalyst compositions (catalysts and/or catalyst systems) disclosed herein can be capable of producing ethylene polymers having an Mw from 40,000 g/mol, 70,000 g/mol, 90.000 g/mol, or 100,000 g/mol to 200,000 g/mol, 300.000 g/mol, 600,000 g/mol, 1,000,000 g/mol, or 1,500,000 g/mol. The ethylene polymers can have a melt index (MI) of 0.6 or greater g/10 min, such as 0.7 or greater g/10 min, such as 0.8 or greater g/10 min, such as 0.9 or greater g/10 min, such as 1.0 or greater g/10 min, such as 1.1 or greater g/10 min, such as 1.2 or greater g/10 min.

“Catalyst productivity” is a measure of how many grams of polymer (P) are produced using a polymerization catalyst comprising W grams of catalyst (cat), over a period of time of T hours; and can be expressed by the following formula: P/(T×W) and expressed in units of gPgcat⁻¹hr⁻¹. The productivity of the catalyst compositions disclosed herein can be at least 50 g(polymer)/g(cat)/hour, such as 500 or more g(polymer)/g(cat)/hour, such as 800 or more g(polymer)/g(cat)/hour, such as 5.000 or more g(polymer)/g(cat)/hour, such as 6,000 or more g(polymer)/g(cat)/hour.

### Process for Making Slurry Catalyst Mixture

A container or vessel can be used to produce or otherwise make the slurry catalyst mixture. One or more mineral oils can be introduced into the vessel. The mineral oil can be heated within the vessel to a temperature of 30° C. to 100° C. Alternatively, the mineral oil can be heated to a temperature ranging from 30° C. to 40° C., from 40° C. to 50° C., from 50° C. to 60° C., from 60° C. to 80° C., from 80° C. to 100° C. or any ranges therebetween, to produce a heated mineral oil. A moisture concentration of the heated mineral oil can be reduced to produce a dried mineral oil. For instance, moisture concentration of the heated mineral oil can be reduced by at least one of: (i) passing a first inert gas through the heated mineral oil, (ii) passing a second inert gas through a headspace of the vessel, (iii) subjecting the heated mineral oil to a vacuum, and (iv) adding an aluminum-containing compound to the heated mineral oil. In various embodiments, two or more, three or more, or four or more of the above may be employed in combination; for instance, a combination of (i) and (ii) may be employed per some embodiments; and/or a combination of (iii) and (iv) in particular embodiments.

Regarding (i) and (ii), the first and/or second inert gases can independently be or include, but are not limited to, nitrogen, carbon dioxide, argon, or any mixture thereof. Amounts of first and/or second inert gas (passed through the mineral oil or into the head space of the vessel) can be gauged in terms of volumetric turnovers (where each turnover equals the volume of the vessel), and can range from a low of 5, 10, 15, or 20 volumetric turnovers to a high of 30, 40, 45, 50, 55, or 60 volumetric turnovers (with ranges from any low to any high contemplated). Vessel volume is not limited but may, for instance, range from a low of any one of 0.75, 1.15, 1.5, 1.9, or 2.3 cubic meters (m³) to a high of 3, 3.8, 5.7, or 7.6 m³. Regarding (iii), the heated mineral oil can be subjected to a vacuum (e.g., a pressure of less than 101 kPa-absolute, less than 75 kPa-absolute, less than 60 kPa-absolute, or less than 55 kPa-absolute). Vacuum pressures in various embodiments may range from a low of any one of 0.67, 1, 10, 15, or 20 kPa-absolute to a high of any one of 30, 40, 55, 60, 65, or 80 kPa-absolute, with ranges from any foregoing low end to any foregoing high end contemplated herein. The heated mineral oil can be subjected to the vacuum for at time period of 1 hour (hr), 2 hr, 3 hr, 4 hr, or 5 hr to 6 hr, 8 hr, 10 hr, 12 hr, 24 hr, or longer. Regarding (iv), the aluminum-containing compound can be or can include, but is not limited to, a compound represented by the formula AlR₍₃₋ₐ₎Xₐ, where R is a branched or straight chain alkyl, cycloalkyl, heterocycloalkyl, aryl, or a hydride radical having from 1 to 30 carbon atoms, X is a halogen, and a is 0, 1, or 2. For instance, the aluminum-containing compound can be or can include tri-hexyl-aluminum, triethylaluminum, trimethylaluminum, tri-isobutylaluminum, di-isobutylaluminum bromide, di-isobutylaluminum hydride, methyl aluminoxane, modified methyl aluminoxane, ethylaluminoxane, isobutylaluminoxane, or any mixture thereof. The modified methyl aluminoxane can be produced by the hydrolysis of trimethylaluminum and a higher trialkylaluminum, such as triisobutylaluminum. Modified methyl aluminoxanes are generally more soluble in aliphatic solvents and more stable during storage. There are a variety of well-known processes for preparing aluminoxanes and modified aluminoxanes.

The moisture concentration of the dried mineral oil can be less than or equal to 100 parts-per-million-water (ppmw), less than or equal to 85 ppmw, less than or equal to 70 ppmw, less than or equal to 60 ppmw, less than or equal to 55 ppmw, less than or equal to 50 ppmw, less than or equal to 45 ppmw, less than or equal to 40 ppmw, less than or equal to 35 ppmw, less than or equal to 30 ppmw, less than or equal to 25 ppmw, or less than or equal to 20 ppmw, as measured according to ASTM D1533-12. The dried mineral oil can have a density of 0.85 g/cm³, 0.86 g/cm³, or 0.87 g/cm³ to 0.88 g/cm³, 0.89 g/cm³, or 0.9 g/cm³ at 25° C., according to ASTM D4052-18a, and/or the dried mineral oil can have a kinematic viscosity at 40° C. of 50 centistokes (cSt), 75 cSt. or 100 cSt to 150 cSt, 200 cSt, 250 cSt. or 300 cSt, according to ASTM D341-20e1. Optionally, the dried mineral oil can also or instead have an average molecular weight of 250 g/mol, 300 g/mol, 350 g/mol, 400 g/mol, 450 g/mol, or 500 g/mol to 550 g/mol, 600 g/mol, 650 g/mol, 700 g/mol, or 750 g/mol, according to ASTM D2502-14(2019)e1.

Once the dried mineral oil has been produced, catalyst particles can be introduced into the dried mineral oil to produce a mixture. The mixture can be mixed, blended, stirred, or otherwise agitated for at least 2 hours to remove at least a portion of any gas that can be present within the pores of the catalyst particles, to produce the slurry catalyst mixture. In some embodiments, the mixture can be agitated for 2 hours, 2.5 hours, 3 hours, 3.5 hours, 4 hours, 4.5 hours, 5 hours, or more to produce the slurry catalyst mixture; and/or the temperature of the mixture can be maintained at a temperature of 50° C. 55° C. 60° C. or 65° C. to 75° C. 80° C., 85° C. or 90° C. during agitation of the mixture. In other embodiments, the temperature of the mixture can be allowed to cool down. For example, the mixture can be allowed to cool down to a temperature of 45° C., 40° C. 35° C., or 30° C. during agitation of the mixture.

The vessel can include one or more mixing apparatus that can be configured to mix, blend, stir, or otherwise agitate the mixture within the vessel. For instance, the mixing apparatus can be a rotatable mixing apparatus. Suitable rotatable mixing apparatus can include one or more blades or impellers configured to agitate one or more components of the slurry catalyst mixture within the vessel when rotated. The rotatable mixing apparatus can be rotated at 40 rotations per minute (rpm), 50 rpm, 75 rpm, or 100 rpm to 150 rpm, 175 rpm, 200 rpm, 225 rpm, or 250 rpm. As another example, the mixture can be agitated via ultrasonic waves; and/or it can be agitated by moving the vessel, e.g., rolling the vessel or rotating the vessel back and forth along an axis thereof.

The mineral oil may also be agitated during introduction into the vessel; during heating of the mineral oil; during reduction of the moisture concentration in the mineral oil; and/or during introduction of the catalyst particles to the mineral oil.

The mineral oil in the slurry catalyst mixture can also be referred to as a diluent. Further, in addition to the mineral oil, the slurry catalyst mixture can also optionally include one or more additional diluents. Additional diluents can be or can include, but are not limited to, toluene, ethylbenzene, xylene, pentane, hexane, heptane, octane, other hydrocarbons, or any combination thereof.

The slurry catalyst mixture can have a solids content of 1 wt. % to 40 wt. %. For instance, the slurry catalyst mixture can have a solids content ranging from 1 wt. % to 5 wt. %, from 5 wt. % to 10 wt. %, from 10 wt. % to 15 wt. %, from 15 wt. % to 20 wt. %, from 20 wt. % to 25 wt. %, from 25 wt. % to 30 wt. %, from 35 wt. % to 40 wt. %, or any ranges therebetween.

Although wax has heretofore been considered necessary for many slurry catalyst mixtures, e.g., for stability (especially for storage and transport), it is noted that slurry catalyst mixtures of various embodiments herein may advantageously omit the wax. Thus, according to such embodiments, the slurry catalyst mixture can be free of any wax having a melting point, at atmospheric pressure, of greater than or equal to 25° C. based on a total weight of the slurry catalyst mixture. More generally, the slurry catalyst mixture can include less than or equal to 3 wt. %, less than or equal to 2.5 wt. %, less than or equal to 2 wt. %, less than or equal to 1.5 wt. %, less than or equal to 1 wt. %, less than or equal to 0.9 wt. %, less than or equal to 0.8 wt. %, less than or equal to 0.7 wt. %, less than or equal to 0.6 wt. %, less than or equal to 0.5 wt. %, less than or equal to 0.4 wt. %, less than or equal to 0.3 wt. %, less than or equal to 0.2 wt %, or less than or equal to 0.1 wt. % of any wax having a melting point, at atmospheric pressure, of greater than or equal to 25° C. based on a total weight of the slurry catalyst mixture. As used herein, the term “wax” includes a petrolatum also known as petroleum jelly or petroleum wax. Petroleum waxes include paraffin waxes and microcrystalline waxes, which include slack wax and scale wax. The wax, if present, can have a density (at 100° C.) of 0.7 g/cm³, 0.73 g/cm³, or 0.75 g/cm³ to 0.87 g/cm³, 0.9 g/cm³, or 0.95 g/cm³. The wax, if present, can have a kinematic viscosity at 100° C. of 5 cSt, 10 cSt. or 15 cSt to 25 cSt, 30 cSt, or 35 cSt. The wax, if present, can have a melting point, at atmospheric pressure, of 25° C., 35° C., or 50° C. to 80° C., 90° C. or 100° C. The wax, if present can have a boiling point of 200° C.′ or greater, 225° C. or greater, or 250° C. or greater.

The term “wax” also refers to or otherwise includes any wax not considered a petroleum wax, which include animal waxes, vegetable waxes, mineral fossil or earth waxes, ethylenic polymers and polyol ether-esters, chlorinated naphthalenes, and hydrocarbon type waves. Animal waxes can include beeswax, lanolin, shellac wax, and Chinese insect wax. Vegetable waves can include carnauba, candelilla, bayberry, and sugarcane. Fossil or earth waxes can include ozocerite, ceresin, and montan. Ethylenic polymers and polyol ether-esters include polyethylene glycols and methoxypolyethylene glycols. The hydrocarbon type waves include waxes produced via Fischer-Tropsch synthesis.

Once the slurry catalyst mixture has been produced, the slurry catalyst mixture can be transferred from the vessel into a catalyst pot or cat pot configured to introduce the slurry catalyst mixture into a gas phase polymerization reactor, such as the gas phase polymerization reactor described in FIG. 1. As such, in various embodiments, the vessel can be located on-site at a manufacturing facility that includes a gas phase polymerization reactor. By making the slurry catalyst mixture on-site at the manufacturing facility, the use of slurry catalyst cylinders to transport the slurry catalyst mixture can be avoided because the slurry catalyst mixture, upon preparation, can be introduced into the catalyst pot or “cat pot” from which the slurry catalyst mixture can be introduced into the gas phase polymerization reactor. By making the slurry catalyst mixture on-site at the manufacturing facility, the slurry catalyst mixture can be introduced into the gas phase polymerization reactor within a time period of less than or equal to 180 minutes, less than or equal to 150 minutes, less than or equal to 125 minutes, less than or equal to 100 minutes, less than or equal to 80 minutes, less than or equal to 60 minutes, less than or equal to 50 minutes, or less than or equal to 40 minutes, upon initiation of agitation of the mixture. On the other hand, by making the slurry catalyst mixture on-site at the manufacturing facility, the slurry catalyst mixture can be introduced into the gas phase polymerization reactor within a time period of less than or equal to 180 minutes, less than or equal to 150 minutes, less than or equal to 125 minutes, less than or equal to 100 minutes, less than or equal to 80 minutes, less than or equal to 60 minutes, less than or equal to 50 minutes, or less than or equal to 40 minutes upon ceasing or stopping agitation of the mixture.

Although, as noted above, wax advantageously may be omitted where storage and/or transport stability are not required for certain catalyst mixtures, it was surprisingly discovered that wax and/or additional diluent in certain catalyst mixtures can aid in the polymerization process in certain cases, e.g., depending on identity(ies) of catalyst compound(s) in the slurry catalyst mixture. Thus, surprisingly, even when one would otherwise think that omitting wax or other diluents is desired (e.g., because there is no need for added storage/transportation stability), it is found that certain catalyst slurries should include wax or other diluents. Thus, processes according to various embodiments may include identifying slurry catalyst mixture(s) for which wax and/or additional diluent is desired and including wax in such slurry catalyst mixture(s) (preferably also while not including wax and/or additional diluent in slurry catalyst mixtures where no processing advantage is obtained by the presence of the wax and/or diluent).

Accordingly, polymerization processes per some embodiments can include, at a first time, introducing a carrier gas, one or more olefins, and a first slurry catalyst mixture into a polymerization reactor. The first slurry catalyst mixture can include a contact product of one or more catalysts selected from a first group of catalysts, a first support, a first activator, a first mineral oil, and a way having a melting point, at atmospheric pressure, of greater than or equal to 25° C. The first slurry catalyst mixture can include greater than 1 wt. % of the wax, based on a total weight of the first slurry catalyst mixture. The one or more olefins can be polymerized in the presence of the first catalyst within the polymerization reactor to produce a first polymer product.

Then, at a second time after the first time, a second slurry catalyst mixture can be introduced into the polymerization reactor. This can occur. e.g., as part of a grade transition or the like in a polymer production campaign (e.g., first slurry catalyst mixture may be stopped before, during, or soon after introduction of the second slurry catalyst mixture). The second slurry catalyst mixture can include a contact product of one or more catalysts selected from a second group of catalysts, a second support, a second activator, and a second mineral oil. The one or more catalysts selected from the second group of catalysts are preferably different from the one or more catalysts selected from the first group of catalysts; however, the first and second supports, activators, and/or mineral oils can be the same or different. The second slurry catalyst mixture, contrary to the first slurry catalyst mixture, can be free of or include less than or equal to 1 wt % of any wax having a melting point, at atmospheric pressure, of greater than or equal to 25° C., based on a total weight of the slurry catalyst mixture. The second slurry catalyst mixture can in particular be produced according to process described above, entailing removing of moisture from the slurry catalyst mixture. The polymerization process can also include polymerizing the one or more olefins in the presence of the second catalyst within the polymerization reactor to produce a second polymer product. The carrier gas can be or can include, but is not limited to, nitrogen, argon, ethane, propane, or any mixture thereof. The one or more olefins can be or can include one more substituted or unsubstituted C₂ to C₄₀ alpha-olefins, as further described below.

In particular, it is believed that catalysts in the first group of catalysts and those of the second group of catalysts will generally have different bulk densities, which aids in identifying which slurry catalyst mixtures may benefit from wax and/or additional diluent, and which will not. For instance, the one or more catalysts in the first group of catalysts can have a bulk density of greater than or equal to 0.43 g/cm³, greater than or equal to 0.44 g/cm³, or greater than or equal to 0.45 g/cm³. On the other hand, the one or more catalysts in the second group of catalysts can have a bulk density of less than 0.45 g/cm³, less than 0.44 g/cm³, less than 0.43 g/cm³, less than 0.42 g/cm³, less than 0.41 g/cm³, or less than 0.40 g/cm³. Put in other words, in various embodiments, the bulk density of the one or more catalyst in the first group of catalysts is greater than the bulk density of the one or more catalysts in the second group of catalysts.

### Catalyst Particles

The catalyst or catalyst compounds can be or can include, but are not limited to, one or more metallocene catalyst compounds. In some embodiments, the catalyst can include at least a first metallocene catalyst compound and a second metallocene catalyst compound, where the first and second metallocene catalyst compounds have different chemical structures from one another. Metallocene catalyst compounds can include catalyst compounds having one or more Cp ligands (cyclopentadienyl and ligands isolobal to cyclopentadienyl) bound to at least one Group 3 to Group 12 metal atom, and one or more leaving group(s) bound to the at least one metal atom. In further embodiments, the catalyst further includes a third and/or a fourth metallocene catalyst compound where the third and fourth metallocene catalyst compounds have different chemical structures from one another and different chemical structures than the first and second metallocene catalyst compounds.

Also suitable are catalyst systems employing a mix of two metallocene catalysts, and in particular, a mix of (1) a bis-cyclopentadienyl hafnocene (preferably a bridged bis-cyclopentadienyl hafnocene) and (2) a zirconocene, such as an indenyl-cyclopentadienyl zirconocene (preferably an unbridged indenyl-cyclopentadienyl zirconocene).

In some embodiments, the metallocene catalyst compounds include a hafnocene. Suitable hafnocenes can include bridged or unbridged hafnocenes, preferably bridged hafnocenes, such as bis(n-propylcyclopentadienyl)hafnium dichloride, bis(n-propylcyclopentadienyl)hafnium dimethyl, (n-propylcyclopentadienyl, pentamethylcyclopentadienyl)hafnium dichloride, (n-propylcyclopentadienyl, pentamethylcyclopentadienyl)hafnium dimethyl, (n-propylcyclopentadienyl, tetramethylcyclopentadienyl)hafnium dichloride, (n-propylcyclopentadienyl, tetramethylcyclopentadienyl)hafnium dimethyl, bis(cyclopentadienyl)hafnium dimethyl, bis(n-butylcyclopentadienyl)hafnium dichloride, bis(n-butylcyclopentadienyl)hafnium dimethyl, and bis(1-methyl-3-n-butylcyclopentadienyl)hafnium dimethyl, and combinations thereof.

Other suitable hafnocene compounds include, but are not limited to, rac/meso Me₂Si(Me₃SiCH₂Cp)₂HfMe₂; rac/meso Me₂Si(Me₃SiCH₂Cp)₂HfMe₂; rac/meso Ph₂Si(Me₃SiCH₂Cp)₂HfMe₂; rac/meso (CH₂)₃Si(Me₃SiCH₂Cp)₂HfMe₂; rac/meso (CH₂)₄Si(Me₃SiCH₂Cp)₂HfMe₂; rac/meso (C₆F₅)₂Si(Me₃SiCH₂Cp)₂HfMe₂; rac/meso (CH₂)₃Si(Me₃SiCH₂Cp)₂ZrMe₂; rac/meso Me₂Ge(Me₃SiCH₂Cp)₂HfMe₂; rac/meso Me₂Si(Me₂PhSiCH₂Cp)₂HfMe₂; rac/meso Ph₂Si(Me₂PhSiCH₂Cp)₂HfMe₂; Me₂Si(Me₄Cp)(Me₂PhSiCH₂Cp)HfMe₂, and combinations thereof.

As noted above, suitable catalyst compounds additionally or alternatively may include a zirconocene, such as an unbridged zirconocene including, but not limited to, bis(indenyl)zirconium dichloride, bis(indenyl)zirconium dimethyl, bis(tetrahydro-1-indenyl)zirconium dichloride, bis(tetrahydro-1-indenyl)zirconium dimethyl, rac/meso-bis(1-ethylindenyl)zirconium dichloride, rac/meso-bis(1-ethylindenyl)zirconium dimethyl, rac/meso-bis(1-methylindenyl)zirconium dichloride, rac/meso-bis(1-methylindenyl)zirconium dimethyl, rac/meso-bis(1-propylindenyl)zirconium dichloride, rac/meso-bis(1-propylindenyl)zirconium dimethyl, rac/meso-bis(1-butylindenyl)zirconium dichloride, rac/meso-bis(1-butylindenyl)zirconium dimethyl, meso-bis(1ethylindenyl)zirconium dichloride, meso-bis(1-ethylindenyl)zirconium dimethyl, (I-methylindenyl) (pentamethyl cyclopentadienyl)zirconium dichloride, (1-methylindenyl) (pentamethyl cyclopentadienyl) zirconium dimethyl, and combinations thereof.

### Slurry Catalyst Mixture Including Activators and Supports

As noted above, the slurry catalyst mixture can include one or more activators and/or supports in addition to one or more catalysts. The term “activator” refers to any compound or combination of compounds, supported or unsupported, which can activate a single site catalyst compound or component, such as by creating a cationic species of the catalyst component. For example, this can include the abstraction of at least one leaving group (the ‘X’ group in the single site catalyst compounds described herein) from the metal center of the single site catalyst compound/component. The activator may also be referred to as a “co-catalyst”. For example, the slurry catalyst mixture can include two or more activators (e.g., aluminoxane and a modified aluminoxane) and a catalyst compound, or the slurry catalyst mixture can include a supported activator and more than one catalyst compound. In particular embodiments, the slurry catalyst mixture can include at least one support, at least one activator, and at least two catalyst compounds. For example, the slurry can include at least one support, at least one activator, and two different catalyst compounds that can be added separately or in combination to produce the slurry catalyst mixture. For instance, a mixture of a support (e.g., silica), and an activator (e.g., aluminoxane) can be contacted with a first catalyst compound, allowed to react, and thereafter the mixture can be contacted with second, different, catalyst compound, for example, in a trim system. And, additional catalyst compounds (third, fourth, etc) could be contacted in a similar manner, in series or together with the first and/or second catalyst compounds.

The molar ratio of metal in the activator to metal in the catalyst compound in the slurry catalyst mixture can be 1000:1 to 0.5:1, 300:1 to 1:1, 100:1 to 1:1, or 150:1 to 1:1. The slurry catalyst mixture can include a support material which can be any inert particulate carrier material known in the art, including, but not limited to, silica, fumed silica, alumina, clay, talc or other support materials, such as disclosed above. In one embodiment, the slurry can include silica and an activator, such as methyl aluminoxane (“MAO”), modified methyl aluminoxane (“MMAO”), as discussed further below. In embodiments, activators include aluminoxane compounds, modified aluminoxane compounds, and ionizing anion precursor compounds that abstract a reactive. σ-bound, metal ligand, making the metal compound cationic and providing a charge-balancing non-coordinating or weakly coordinating anion.

As noted above, one or more organo-aluminum compounds, such as one or more alkylaluminum compounds, can be used in conjunction with the aluminoxanes. For example, alkylaluminum species that can be used include diethylaluminum ethoxide, diethylaluminum chloride, and/or disobutylaluminum hydride. Examples of trialkylaluminum compounds include, but are not limited to, trimethylaluminum, triethylaluminum (“TEAL”), triisobutylaluminum “TiBAl”), tri-n-hexylaluminum, tri-n-octylaluminum, tripropylaluminum, tributylaluminum, and the like.

Suitable supports include, but are not limited to, active and inactive materials, synthetic or naturally occurring zeolites, as well as inorganic materials such as clays and/or oxides such as silica, alumina, zirconia, titania, silica-alumina, cerium oxide, magnesium oxide, or combinations thereof. In particular, the support may be silica-alumina, alumina and/or a zeolite, particularly alumina. Silica-alumina may be either naturally occurring or in the form of gelatinous precipitates or gels including mixtures of silica and metal oxides.

In some embodiments, at least a portion of the slurry catalyst mixture can be contacted with a solution catalyst mixture to produce or otherwise form a slurry/solution catalyst mixture.

### Solution Catalyst Mixture—Trim Solution

The solution catalyst mixture can include a solvent and only catalyst compound(s), such as one or more metallocene catalyst compounds, or can also include an activator. In some embodiments employing two catalyst compounds, the solution catalyst mixture can be or can include, but is not limited to, a contact product of a solvent/diluent and the first catalyst or the second catalyst compounds. In embodiments employing more than two catalyst compounds, the solution catalyst mixture can include the contact product of solvent/diluent and any one or more of the first, second, third, etc. catalyst compounds. The catalyst compound(s) in the solution catalyst mixture can be unsupported. Further, the slurry/solution catalyst mixture can be introduced into the gas phase polymerization reactor.

The solution catalyst mixture, if used, can be prepared by dissolving the catalyst compound(s) and optional activators in a liquid solvent. The liquid solvent can be an alkane, such as a C₅ to C₃₀ alkane, or a C₅ to C₁₀ alkane. Cyclic alkanes (e.g., cyclohexane) and aromatic compounds (e.g., toluene) can also be used. Mineral oil can be used as a solvent alternatively or in addition to other alkanes such as one or more C₅ to C₃₀ alkanes. The mineral oil in the solution catalyst mixture, if used, can have the same properties as the mineral oil that can be used to make the slurry catalyst mixture, as described above. The solvent should be liquid under the conditions of polymerization and relatively inert. Optionally, the solvent utilized in the solution catalyst mixture can be different from the diluent used in the slurry catalyst mixture. Or, on the other hand, the solvent utilized in the solution catalyst mixture can be the same as the diluent. i.e., the mineral oil(s) and any additional diluents used in the slurry catalyst mixture.

If the solution catalyst mixture includes both the catalyst compound(s) and an activator, the ratio of metal in the activator to metal in all catalyst compound(s) in the solution catalyst mixture can be 1000:1 to 0.5:1, 300:1 to 1:1, or 150:1 to 1:1. In various embodiments, the activator and catalyst compound(s) can, together, be present in the solution catalyst mixture at up to 90 wt. %, at up to 50 wt. %, at up to 20 wt. %, such as at up to 10 wt. %, at up to 5 wt. %, at less than 1 wt. %, or between 100 ppm and 1 wt. %, based on the weight of the solvent, the activator, and the catalyst. The one or more activators in a solution catalyst mixture, if used, can be the same or different as the one or more activators used in a slurry catalyst mixture.

The solution catalyst mixture can include any one of the catalyst compound(s) of the present disclosure. As the catalyst is dissolved in the solution, a higher solubility can be desirable. Accordingly, the catalyst in the solution catalyst mixture can often include a metallocene, which may have higher solubility than other catalysts. In the polymerization process, any of the above described solution catalyst mixtures can be combined with any of the slurry catalyst mixtures described above. In addition, more than one solution catalyst mixture can be utilized.

### Continuity Additive—Static Control Agent

In gas-phase polyethylene production processes, it can be desirable to use one or more static control agents to help facilitate the regulation of static levels within the reactor. A continuity additive is a chemical composition that, when introduced into the fluidized bed within the reactor, can influence or drive a static charge (negative, positive, or to zero) in the fluidized bed. The continuity additive used can depend, at least in part, on the nature of the static charge, and the choice of static control agent can vary depending, at least in part, on the polymer being produced and/or the single site catalyst compounds being used. In some embodiments, the continuity additive or static control agent can be introduced into the reactor in an amount of 0.05 ppm to 200 ppm. Alternatively, the amount of the continuity additive or static control agent may range from 0.05 ppm to 2 ppm, 5 ppm, or 10 ppm, or range from 20 ppm to 50 ppm, 75 ppm, 100 ppm, 150 ppm, or 200 ppm.

In some embodiments, the continuity additive can be or can include aluminum stearate. The continuity additive can be selected for its ability to receive the static charge in the fluidized bed without adversely affecting productivity. Other suitable continuity additives can be or can include, but are not limited to, aluminum distearate, ethoxylated amines, and combinations thereof. In some embodiments, the continuity additive includes a mixture of a polysulfone copolymer, a polymeric polyamine, and oil soluble sulfonic acid. Any of the continuity additives can be used either alone or in combination.

In some embodiments, the continuity additive can include fatty acid amines, amide-hydrocarbon or ethyoxylated-amide compounds, carboxylate compounds such as aryl-carboxylates and long chain hydrocarbon carboxylates, and fatty acid-metal complexes; alcohols, ethers, sulfate compounds, metal oxides and other compounds known in the art. Some specific examples of control agents can be or can include, but are not limited to, 1,2-diether organic compounds, magnesium oxide, glycerol esters, ethoxylated amines (e.g. N,N-bis(2-hydroxyethyl) octadecylamine), alkyl sulfonates, and alkoxylated fatty acid esters, chromium N-oleylanthranilate salts, calcium salts of a Medialan acid and di-tert-butylphenol, an α-olefin-acrylonitrile copolymer and polymeric polyamine, sorbitan-monooleate, glycerol monostearate, methyl toluate, dimethyl maleate, dimethyl furnarate, triethylamine, 3,3-diphenyl-3-(imidazol-1-yl)-propin, and like compounds. In some embodiments, another continuity additive can include a metal carboxylate salt, optionally, with other compounds.

In some embodiments, the continuity additive can include an extracted metal carboxylate salt that can be combined with an amine containing agent, such an extracted carboxy late metal salt. For example, the extracted metal carboxylate salt can be combined with antistatic agents such as fatty amines, such as a blend of ethoxylated stearyl amine and zinc stearate, or a blend of ethoxylated stearyl amine, zinc stearate and octadecyl-3,5-di-tert-butyl-4-hydroxyhydrocinnamate.

Other continuity additives can include ethyleneimine additives, such as polyethyleneimines having the following general formula: —(CH₂—CH₂—NH)n-, where n can be from 10 to 10,000. The polyethyleneimines can be linear, branched, or hyper branched (e.g. forming dendritic or arborescent polymer structures). The polyethyleneimines can be a homopolymer or copolymer of ethyleneimine or mixtures thereof (referred to as polyethyleneimine(s) hereafter). Although linear polymers represented by the chemical formula —(CH₂)—CH₂—NH)n- can be used as the polyethyleneimine, materials having primary, secondary, and tertiary branches can also be used.

### Induced Condensing Agent

In gas-phase polyethylene production processes, it can be desirable to use one or more induced condensing agents within the reactor. “Induced condensing agent (ICA),” as used herein, refers to one or more induced condensable fluids, which are readily volatile liquid hydrocarbons that may be selected from saturated hydrocarbons containing from 2 to 10 carbon atoms, preferably 3 to 10 carbon atoms. Some suitable saturated hydrocarbons are propane, n-butane, isobutane, n-pentane, isopentane, neopentane, n-hexane, isohexane, and other saturated C₆ hydrocarbons, n-heptane, n-octane and other saturated C₇ and C₈ hydrocarbons or mixtures thereof. A class of preferred induced condensable hydrocarbons includes C₅ and C₆ saturated hydrocarbons. Another class of preferred hydrocarbons includes C₄ to C₆ saturated hydrocarbons. Preferred hydrocarbons for use as condensable fluids include pentanes, such as isopentane. The condensable fluids may also include polymerizable condensable comonomers such as olefins, diolefins or mixtures thereof, including some of the monomers mentioned herein, which may be partially or entirely incorporated in the polymer product.

### End Uses

The polymers produced by the processes disclosed herein and blends thereof can be useful in forming operations, such as film, sheet, and fiber extrusion and co-extrusion as well as blow molding, injection molding, and rotary molding. Films include blown or cast films formed by co-extrusion or by lamination useful as shrink film, cling film, stretch film, sealing films, oriented films, snack packaging, heavy duty bags, grocery sacks, baked and frozen food packaging, medical packaging, industrial liners, membranes, etc., in food-contact and non-food contact applications. Fibers include melt spinning, solution spinning and melt blown fiber operations for use in woven or non-woven form to make filters, diaper fabrics, medical garments, geotextiles, etc. Extruded articles include medical tubing, wire and cable coatings, pipe, geomembranes, and pond liners. Molded articles include single and multi-layered, constructions in the form of bottles, tanks, large hollow articles, rigid food containers and toys, etc.

Specifically, any of the foregoing polymers, such as ethylene copolymers or blends thereof, can be used in mono- or multi-laver blown, extruded, and/or shrink films. These films may be formed by any number of well-known extrusion or coextrusion techniques, such as a blown bubble film processing technique, wherein the composition can be extruded in a molten state through an annular die and then expanded to form a uni-axial or biaxial orientation melt prior to being cooled to form a tubular, blown film, which can then be axially slit and unfolded to form a flat film. Films may be subsequently unoriented, uniaxially oriented, or biaxially oriented to the same or different extents.

The polymers produced herein may be further blended with one or more second polymers and used in film, molded parts, and other typical applications. In one embodiment, the second polymer can be selected from ethylene homopolymer, ethylene copolymers, and blends thereof. Useful second ethylene copolymers can include one or more comonomers in addition to ethylene, and can be a random copolymer, a statistical copolymer, a block copolymer, and/or blends thereof. The process of making the second ethylene polymer is not limited, as it can be made by slurry, solution, gas phase, high pressure or other suitable processes, and by using catalyst systems appropriate for the polymerization of polyethylene, such as Ziegler-Natta-type catalysts, chromium catalysts, metallocene-type catalysts, other appropriate catalyst systems or combinations thereof, or by free-radical polymerization.

### Dual-Component Catalyst System in Slurry

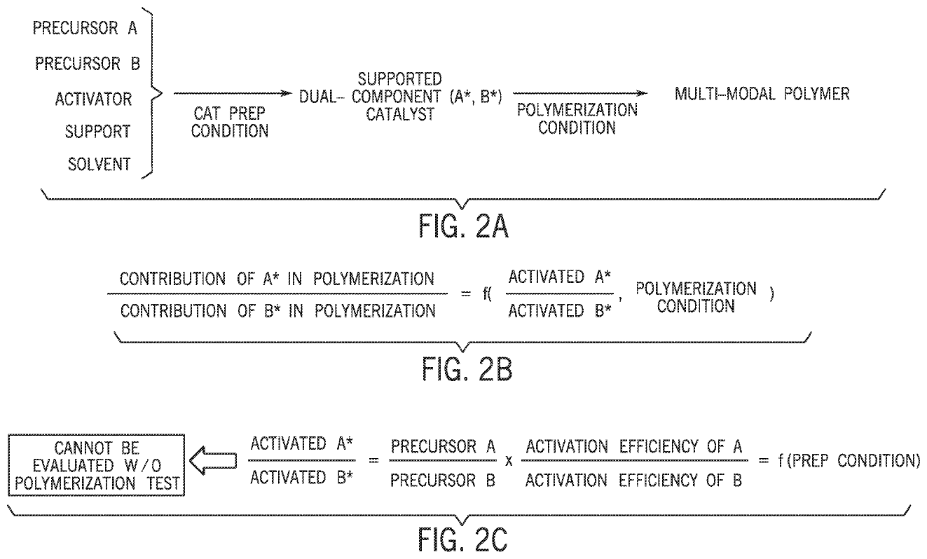

As discussed above, the mole ratio of the various (e.g., at least two) types of active sites (e.g., deposited and supported catalyst compounds) on the dual-component catalyst system do not necessarily equal the mole ratio of the catalyst compound precursors used in the preparation of the supported catalyst system due to the different activation energies and activation efficiencies. FIG. 2A illustrates an example method of producing a dual-component catalyst system (labeled in FIG. 2 as a “Dual Component Catalyst”) and using the dual-component catalyst system in a production of a multi-modal polymer. FIG. 2B illustrates the contribution in polymerization of the two types of active sites (supported active catalyst compounds) in a dual-component catalyst system. FIG. 2C illustrates the relationship between the mole ratio of the two types of active sites in the dual-component catalyst system and the mole ratio of the two type of catalyst compound precursors used in making the dual-component catalyst system. From FIGS. 2A-C, it is apparent that the relative contribution in polymerization between the two types of active sites (which in turn could be used to determine composition of the resulting multi-modal polymer) depends on the molar ratio of the two types of active sites (catalyst compounds) on the support in the dual-component catalyst system. The assessment of the mole ratio of the two types of active sites in the dual-component catalyst system has heretofore relied solely on polymerization testing (e.g., back-calculating the mole ratio of the two active sites based upon properties of the produced polymer).

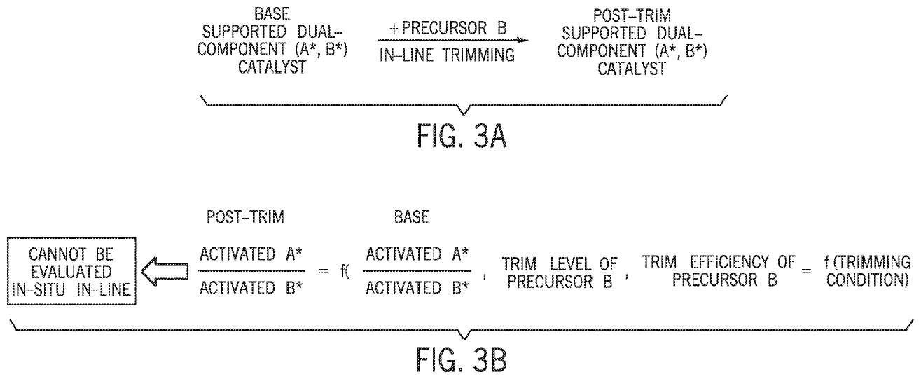

One example of in-line trimming is described above in connection with FIG. 1. The in-line trimming process allows a base supported catalyst system (comprising the contact product of support, optional activator(s), and one, two, or more catalyst compounds) to react with one, two, or more additional catalyst compounds (which can be the same or different from the catalyst compound(s) in the base supported catalyst system), yielding a post-trim catalyst that features a different ratio of the two or more catalyst compounds (that is, the two or more types of active sites on the dual-component catalyst system) prior to entering the gas phase reactor. Frequently, it will be preferred to have a base supported catalyst system having two (or three, etc.) catalyst compounds deposited thereon (that is, two, three, etc. active sites) contacted with additional catalyst compound of one type among the two (or more) catalyst compounds deposited on the base supported catalyst system; thus enabling adjustment of the ratio of deposited catalyst compounds on the support in the post-trim catalyst system. FIG. 3A illustrates a production scheme for a post-trim catalyst system. FIG. 3B illustrates that the relative ratio of the two types of active sites A* and B* in a post-trim catalyst system, depends on the original ratio in base catalyst system, the trim level (the amount of precursor B trimmed per gram of base catalyst system), and the trim efficiency. In this example. “trim efficiency” is the percentage of trimmed precursor B that was activated; more generally, “trim efficiency” is the percentage of trimmed catalyst compound(s) (i.e., those added by contacting a catalyst slurry with a catalyst trim solution, per the catalyst trimming process described above) that are actually activated (that is, active in the post-trim catalyst system). In general, it is difficult to accurately predict and control the ratio of the active sites in the post-trim catalyst.

It is noted that, although much of the discussion and examples herein focus on a dual-component catalyst system (having two catalyst compounds), it is anticipated that methods and systems described herein could be readily adapted to systems employing three, four, or more catalyst compounds. Thus, references to “dual-component catalyst” herein can more generally be taken as references to a “multi-component catalyst” having 2 or more catalyst compounds deposited on support (that is, two or more types of active sites).

### Soft Sensor to Estimate Trim Effectiveness

With the foregoing in mind, the trim efficiency is an important parameter of multi-component catalyst systems. In particular, trim efficiency substantially impacts the properties of the resulting polymer products, such as density, melt index (MI), melt index ratio (MIR), stiffness, toughness, and processibility. However, there are currently no established or commercially available analytical methods (e.g., hardware-sensor-based methods) to directly measure trim efficiency for a multi-component catalyst system. As such, present embodiments are directed to a soft sensor system that enables the determination of a catalyst trim effectiveness value (η) for a multi-component catalyst system (e.g., a dual-component catalyst system), wherein the catalyst trim effectiveness is an estimated or approximated value for the actual trim efficiency within the multi-component catalyst system. Accordingly, the catalyst trim effectiveness value (η) ranges between zero (representing no trim effectiveness) and one (representing total trim effectiveness). As used herein, a “soft sensor” or a “virtual sensor” is a set of computer-executable instructions (e.g., software instructions) that, when executed by a processor of a computing system, are designed to receive a set of inputs, process these inputs using one or more mathematical models, and provide at least one output from the one or more mathematical models. It may be appreciated that, while the discussion below is directed to gas-phase polyethylene (GPPE) polymerization processes and polyethylene (PE) resins as an example, in other embodiments, the techniques disclosed herein may be applicable to other types of polymerization reactors (e.g., slurry-phase polymerization systems), as well as other types of polymer products.

As illustrated in FIG. 1, in certain embodiments, the gas-phase reactor system 100 may include a soft sensor system 160 having suitable computer circuitry to enable the soft sensor techniques disclosed herein. For the embodiment illustrated in FIG. 1, the soft sensor system 160 includes at least one processor 162 (e.g., processing circuitry, a central processing unit (CPU), a graphics processing unit (GPU)), at least one memory 164 (e.g., random access memory (RAM), read-only memory (ROM), non-transitory computer-readable media), and at 20 least one storage 166 (e.g., a solid state disk, a hard drive, a flash drive). As discussed below, the memory 164 and/or storage 166 are designed to store instructions (e.g., software instructions, computer-executable code) executed by the at least one processor 162, as well as data (e.g., inputs, outputs, intermediates), to perform the techniques described herein. In some embodiments, the soft sensor system 160 includes at least one networking device 168 (e.g., a wired or wireless networking interface) that enables the soft sensor system 160 to send and receive data such as receiving inputs from users, receiving information about the configuration or operation of other components of the gas-phase reactor system 100, adjusting the configuration or operation of other components of the gas-phase reactor system 100, providing outputs to users, and so forth. In some embodiments, the soft sensor system 160 may be implemented separately from the gas-phase reactor system 100, such as in a server room, in a data center, or in a cloud-based environment.

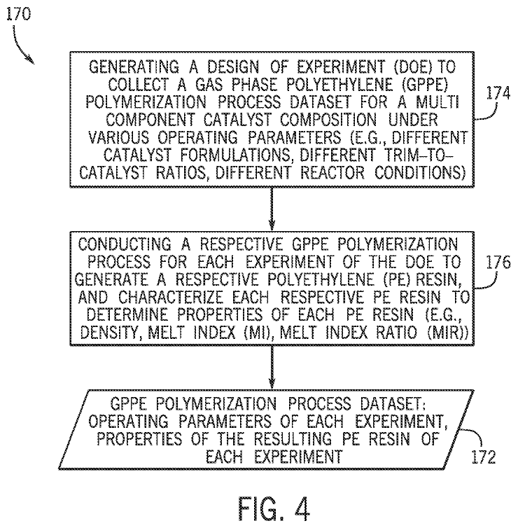

FIG. 4 is a flow diagram of an embodiment of a process 170 of generating a gas-phase polyethylene (GPPE) polymerization process dataset 172. As discussed below, the GPPE polymerization process dataset 172 is used by the soft sensor system 160 to determine respective catalyst trim effectiveness values (η) for a given multi-component catalyst composition. For the illustrated embodiment, the process 170 begins with generating (block 174) a design of experiment (DOE) to collect the GPPE polymerization process dataset 172 for a multi-component catalyst composition (e.g., a dual-component catalyst composition) under various operating parameters of the gas-phase reactor system 100. These operating parameters may include, but are not limited to: different catalyst formulations, different trim-to-catalyst ratios, and different parameters of the gas-phase reactor system 100 (e.g., different mixing temperatures, reaction temperatures, mixing times, reaction times, reactor pressures, flow rates). In general, the DOE seeks to ensure the experimental data of the GPPE polymerization process dataset 172 substantially or entirely covers the high dimensional independent variable space for the multi-component catalyst. In some embodiments, the DOE may be generated using an active learning protocol, while in some embodiments, the DOE may be generated using classical screening, follow up, and response surface experiment designs, or other suitable techniques.

For the embodiment illustrated in FIG. 4, the process 170 continues with conducting (block 174) a respective GPPE polymerization process for each experiment of the DOE to generate a respective polyethylene (PE) resin using the multi-component catalyst composition. Additionally, at block 174, each PE resin is characterized to determine relevant properties of each respective PE resin. The properties of the PE resin may include, but are not limited to: density, melt index (MI), and melt index ratio (MIR), which may be determined as discussed above. In certain embodiments, one or more aspects of performing the GPPE polymerization processes and/or one or more aspects of characterizing the resulting PE resins may be automated for reduced cost and enhanced efficiency. The result or output of the process 170 is the GPPE polymerization process dataset 172, which includes the operating parameters of each experiment of the DOE, as well as the characterized properties of the PE resin yielded by each of these experiments. In certain embodiments, the soft sensor system 160 may receive information regarding the operating parameters of each GPPE polymerization process directly from one or more components of the gas-phase reactor system 100 and/or receive information regarding the characterization of the PE resins directly from the equipment used to perform these analyses, which is then included in the GPPE polymerization process dataset 172.

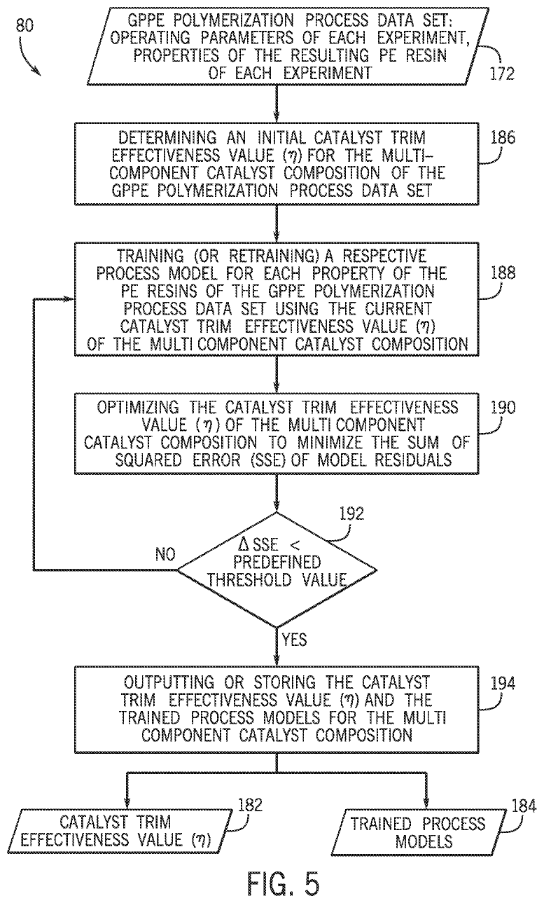

FIG. 5 is a flow diagram of an embodiment of a process 80 of determining a catalyst trim effectiveness value (η) 182 and trained process models 184 for the multi-component catalyst composition associated with the GPPE polymerization process dataset 172. The process 80 may be stored as computer-executable instructions (e.g., software) in the at least one memory 164, and may be executed by the at least one processor 162, of the soft sensor system 160 illustrated in FIG. 1. As illustrated in FIG. 5, the GPPE polymerization process dataset 172, which may be generated as discussed with respect to FIG. 4, is provided as input to the process 80.

For the embodiment illustrated in FIG. 5, the process 80 begins with the processor 162 determining (block 186) (e.g., calculating or assuming) an initial catalyst trim effectiveness value (η) for the multi-component catalyst composition. In some embodiments, the initial catalyst trim effectiveness value (η) of the multi-component catalyst composition may be assumed to be a predefined value (e.g., 0.5). In some embodiments, the processor 162 may calculate the initial catalyst trim effectiveness value (η) of the multi-component catalyst composition based on known catalyst activation fundamentals (e.g., an a priori approach), for example, using Equation 1:

$$η = f ( MAO\_in \_activated \_catalyst , {T}_{ambinet} , {t}_{contact} , Trim\_solvent , ... ) , \qquad Equation 1$$

wherein
MAO_in_activated_catalyst is the amount of the activator, such as MAO or MMAO, present in the activated multi-component catalyst composition (e.g., in grams);
T_{ambinet} is the ambient temperature (e.g., in degrees Celsius);
t_{contact} is the contact time of the multi-component catalyst composition (e.g., in the static mixer 109);
Trim_solvent is the identity of the trim solvent.
   As indicated by the ellipsis in the list of input parameters of the function ƒ for Equation 1, in other embodiments, other inputs related to the multi-component catalyst composition and/or the operating parameters of the gas phase reactor system may also be used, in accordance with the present disclosure. The ordinarily skilled artisan with the benefit of this disclosure will recognize other parameters which could affect trim efficiency (i.e., other catalyst fundamentals) based upon the details of a given process for which the control methods and systems of this disclosure are being deployed.

For the embodiment illustrated in FIG. 5, the process 80 continues with the processor 162 using the current catalyst trim effectiveness value (η) of the multi-component catalyst composition to train (block 188) a respective process model for each property of the PE resins present in the GPPE polymerization process dataset 172. Each process model is trained using a suitable machine learning method, including but not limited to: Elastic net regularization methods, least absolute shrinkage and selection operator (LASSO) methods, Ridge regression methods, and Stepwise regression methods, random forest, gradient boost, neural network, Gaussian process model, support vector machine, multi-variate adaptive regression spline. During training, each process model “learns” relationships between a particular PE resin property of the GPPE polymerization process dataset 172 (e.g., density, MI, MIR), the activated multi-component catalyst composition, and the operating parameters of the GPPE polymerization process. In certain embodiments, an example process model is represented by Equation 2:

$$Property\_of \_Resin = g ( CatComp , T , H - to - M , C - to - M , I C A , A - P P , t ) , \qquad Equation 2$$

wherein
Property_of_Resin is a particular property (e.g., density, MI, MIR) of the PE resin;
CatComp is the activated multi-component catalyst composition;
T is the reactor bed temperature (e.g., in degrees Celsius);
H-to-M is the ratio of hydrogen to alpha-olefin monomer (e.g., ethylene gas) in the reactor;
C-to-M is the ratio of one or more alpha-olefin comonomers (e.g., hexene) to alpha-olefin monomer in the reactor;
ICA is amount (e.g., molar percentage) of induced condensing agent (ICA) (e.g., isopentane, isobutane) in the reactor;
M-PP is the partial pressure (e.g., in kilopascal) of alpha-olefin monomer in the reactor; and
t is the reactor residence time for the PE resin (e.g., in seconds).

In Equation 2, the CatComp value is calculated using the current catalyst trim effectiveness value (η) of the activated multi-component catalyst composition associated with the multi-component catalyst. For example, in a dual-component catalyst composition that includes (e.g., from catalyst pot 106) catalyst A (e.g., a hafnocene catalyst, or a bridged bis-cyclopentadienyl hafnocene catalyst) and catalyst B (e.g., a zirconocene catalyst, or an unbridged indenyl-cyclopentadienyl zirconocene catalyst), wherein catalyst B also serves as the trim catalyst (e.g., from trim solution in trim pot 108), the CatComp of the activated multi-component catalyst composition may be calculated using Equation 3:

$$CatComp = \frac{{A}_{s}}{{A}_{s} + {B}_{s} + {B}_{t} \times η} , \qquad Equation 3$$

wherein
Aₛ is the amount (e.g., moles catalyst) of catalyst A loaded onto the support;
Bₛ is the amount (e.g., moles catalyst) of catalyst B loaded onto the support; and
Bₜ is the amount (e.g., moles catalyst) of catalyst B provided as trim.
η is the trim effectiveness value (already defined) of catalyst B.
   As such, for the example activated dual-component catalyst formulation, the CatComp value may range between zero and one. It may be appreciated that, for other multi-component catalyst systems (e.g., tri-catalyst systems, quad-catalyst systems, penta-catalyst systems), Equation 3 would be modified to reflect the contribution of each of the catalyst components of the multi-component catalyst, in accordance with the present disclosure. For example, the disclosed approach could be used for a tri-catalyst system, in which catalyst A and catalyst B are initially loaded onto the support, and catalyst C (not initially loaded onto the support) is provided as trim.

For the embodiment illustrated in FIG. 5, the process 80 continues with the processor 162 optimizing (block 190) the catalyst trim effectiveness value (η) for the multi-component catalyst composition, with the optimization determined with reference to the goal of minimizing the sum of squared error (SSE) of model residuals. For example, in certain embodiments, the sum of squared error (SSE) value of model residuals may be calculated based on the predicted properties and the measured properties of all of the PE resins fabricated using the multi-component catalyst in accordance with Equation 4:

$$S S E = \sum {( M I {R}_{p} - M I {R}_{m} )}^{2} + \sum {( M {I}_{p} - M {I}_{m} )}^{2} + \sum {( {D}_{p} - {D}_{m} )}^{2} , \qquad Equation 4$$

wherein
MIRₚ is a melt index ratio predicted by the melt index ratio process model of a PE resin;
MIRₘ is a measured melt index ratio of the PE resin;
MIₚ is a melt index predicted by the melt index process model of a PE resin;
MIₘ is a measured melt index of the PE resin;
Dₚ is a density predicted by the density process model of a PE resin; and
Dₘ is the measured density of the PE resin.
   As such, each sigma notation (E) iterates through each PE resin product included in the GPPE polymerization process dataset 172 of the multi-component catalyst, summing the square of the difference between the predicted and measured property values. Since the properties of the PE resins predicted by the process models depend on the catalyst trim effectiveness value (η), as noted above, during the optimization of block 190, the catalyst trim effectiveness value (η) of the multi-component catalyst may be varied (e.g., increased or decreased) until the SSE value reaches a minimum value (e.g., a local minimum), wherein the predicted and measured properties of the PE resins are closest in value for the current version or iteration of the process models. This process of varying the trim effectiveness value (η) of the multi-component catalyst to obtain a minimized SSE value (that is, to minimize the SSE value) is what is meant by “optimizing” the catalyst trim effectiveness value.

For the embodiment illustrated in FIG. 5, the process 80 continues with the processor 162 determining (decision block 192) whether a change (e.g., a decrease) in the minimized SSE value between the present iteration of block 190 and a previous iteration of block 190 (ΔSSE) is less than a predefined threshold value (e.g., 5%, 2%, 1%). In some embodiments, prior to the first iteration of block 190, the previous minimized SSE value may be initialized to a particular value (e.g., zero), such that, any value of SSE determined in the first iteration of block 190 results in a ΔSSE at decision block 192 that is greater than the predefined threshold value. When this occurs, the processor 162 returns to block 188 and proceeds to retrain the process models using the current catalyst trim effectiveness value (η) of each catalyst formulation, then repeats the actions of block 190 to again optimize the catalyst trim effectiveness value (η) to minimize the SSE value calculated using the retrained process models. In the second iteration of decision block 192, the processor 162 determines whether the change between the minimized SSE value determined in the first iteration of block 190 and the minimized SSE value determined m the second iteration of block 190 (ΔSSE) is less than the predefined threshold value. Accordingly, the processor 162 continues to iterate through blocks 188 and 190 while the ΔSSE is greater than the predefined threshold value.

For the embodiment illustrated in FIG. 5, once the processor 162 determines in decision block 192 that the ΔSSE is less than the predefined threshold value, the processor 162 responds by outputting or storing (block 194) the current catalyst trim effectiveness value (η) of the multi-component catalyst composition 182, which may be referred to as the optimized catalyst trim effectiveness value. Additionally, the processor 162 outputs or stores the trained process models 184 (e.g., a density process model, a MI process model, a MIR process model) associated with the multi-component catalyst composition. In certain embodiments, the optimized catalyst trim effectiveness value (η) and the trained process models may be suitably stored in memory 164 and/or storage 166 for later use. It may be appreciated that, as additional PE resins are produced using the multi-component catalyst composition and characterized over time, the GPPE polymerization process dataset 172 may be augmented to include this data, and the process 80 may be repeated to further tune or refine the catalyst trim effectiveness value (η) 182 and the trained process models 184 associated with the multi-component catalyst composition.

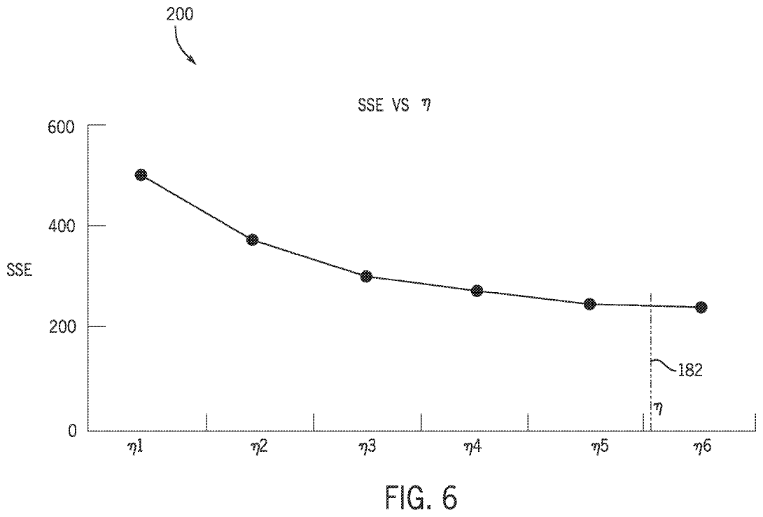

To better illustrate how the SSE values change as the catalyst trim effectiveness value (η) changes. FIG. 6 is a graph 200 illustrating an example plot of minimized SSE values versus the number of iterations through the n optimization process (block 190 of FIG. 5), wherein each iteration results in an updated catalyst trim effectiveness value (e.g., η₁, η₂, η₃, η₄, η₅, η₆). For this example, the minimized SSE value starts at a relative maximum in the first iteration of block 190 (e.g., η₁), and the minimized SSE value diminishes with each subsequent iteration (e.g., η₂, η₃, η₄, η₅, η₆). However, at each iteration of block 190, the decrease in the minimized SSE value also diminishes, such that the ΔSSE is greatest between the first two iterations of block 190 (e.g., between η₁ and η₂), and decreases with each subsequently iteration of block 190. At the sixth iteration, the ΔSSE that results from the final two catalyst trim effectiveness values (e.g., η₅ and η₆), falls below the predefined threshold value discussed with respect to FIG. 5, and the process 80 of FIG. 5 concludes by selecting the catalyst trim effectiveness value determined by the final iteration of block 190 (e.g., no) as the catalyst trim effectiveness value 182 for the multi-component catalyst composition.

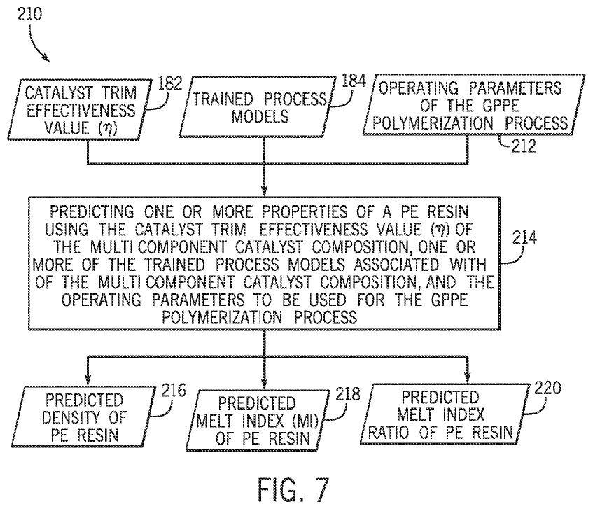

FIG. 7 is a flow diagram illustrating an embodiment of a process 210 whereby the processor 162 may use the catalyst trim effectiveness value (η) 182 and the trained process models 184, as provided by the process 80 of FIG. 5, to predict the properties of a PE resin to be produced using the multi-component catalyst composition. The process 210 is stored as computer-executable instructions (e.g., software) in the at least one memory 164, and may be executed by the at least one processor 162, of the soft sensor system 160 illustrated in FIG. 1.

For the embodiment illustrated in FIG. 7, the processor 162 receives the catalyst trim effectiveness value (η) 182 of the multi-component catalyst composition and the trained process models 184 associated with the multi-component catalyst composition, determined as discussed above. The processor 162 also receives inputs defining various operating parameters 212 of the GPPE polymerization process to be used in preparing the PE resin. In certain embodiments, the soft sensor system 160 may communicate directly with one or more components of the gas-phase reactor system 100 to directly and automatically determine the current configurations or operating parameters of these components.

For the embodiment illustrated in FIG. 7, the processor 162 then predicts (block 214) one or more properties of the PE resin using the catalyst trim effectiveness value (η) 182 of the multi-component catalyst composition, one or more of the trained process models 184, and the operating parameters 212 of the GPPE polymerization process to be used to produce the PE resin. For the illustrated embodiment, the processor 162 outputs a predicted density 216 of the PE resin, a predicted melt index (MI) 218 of the PE resin, and a predicted melt index ratio (MIR) 220 of the PE resin. As such, the embodiments disclosed herein enable predictions of properties of PE resins produced using a multi-component catalyst composition for a given set of operating parameters. It may be appreciated that the process 210 of FIG. 7 is merely provided as an example, and in other embodiments, the processor 162 may use the catalyst trim effectiveness value (η) 182 and the trained process models 184 in different manners to improve the polymerization process.

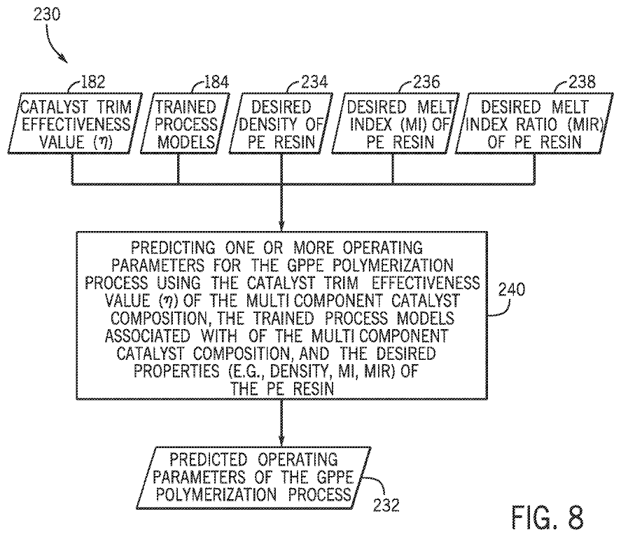

For example. FIG. 8 is a flow diagram illustrating an embodiment of a process 230 whereby the processor 162 may use the catalyst trim effectiveness value (η) 182, the trained process models 184, and desired properties of a PE resin to be produced the multi-component catalyst composition to determine predicted operating parameters 232 for the GPPE polymerization process. The process 230 may be stored as computer-executable instructions (e.g., software) in the at least one memory 164, and may be executed by the at least one processor 162, of the soft sensor system 160 illustrated in FIG. 1.

For the embodiment illustrated in FIG. 8, the processor 162 receives the catalyst trim effectiveness value (η) 182 of the multi-component catalyst composition and the trained process models 184 associated with the multi-component catalyst composition, determined as discussed above, as well as inputs (e.g., user inputs) defining the desired properties of the PE resin. For the illustrated embodiment, inputs include a desired density 234 of the PE resin, a desired melt index (MI) 236 of the PE resin, and a desired melt index ratio (MIR) 238 of the PE resin. The processor 162 then determines (block 240) one or more predicted operating parameters 232 for the GPPE polymerization process using the catalyst trim effectiveness value (η) 182 of the multi-component catalyst composition, the trained process models 184, and the desired properties 234, 236, and 238 (e.g., density, MI, MIR) of the PE resin, wherein the predicted operating parameters 232 are expected to produce a PE resin with the desired properties. In certain embodiments, the soft sensor system 160 may communicate directly with one or more components of the gas-phase reactor system 100 to modify or adjust the current configurations or operating parameters of these components in accordance with the predicted operating parameters 232. As such, the embodiments disclosed herein enable predictions of the operating parameters of the GPPE polymerization to use with a multi-component catalyst composition to yield a PE resin with one or more desired properties.

### ADDITIONAL EMBODIMENTS

Accordingly, the present disclosure may provide a soft sensor system, a method, and a computer-readable medium for determining a catalyst trim effectiveness value of a multi-component catalyst composition. The methods and systems may include any of the various features disclosed herein, including one or more of the following statements.

Statement 1. A soft sensor system, comprising: at least one memory configured to store a polymerization process dataset for a multi-component catalyst composition, wherein the polymerization process dataset includes one or more properties of polyethylene (PE) resins prepared using the multi-component catalyst composition and different operating parameters of a gas phase reactor system; and at least one processor configured to execute stored instruction to perform actions comprising: (A) setting a catalyst trim effectiveness value of the multi-component catalyst composition to an initial value; (B) training a respective process model for each of the one or more properties of the PE resins of the polymerization process dataset using the catalyst trim effectiveness value of the multi-component catalyst composition to yield a set of one or more trained process models; (C) optimizing the catalyst trim effectiveness value of the multi-component catalyst composition to minimize a sum of squared error (SSE) value of model residuals based on the set of trained process models; (D) repeating steps (B) and (C) until a decrease in the minimized SSE value is less than a predefined threshold value, and (E) outputting or storing the optimized catalyst trim effectiveness value and the set of one or more trained process models for the multi-component catalyst composition.

Statement 2. The soft sensor system of statement 1, wherein, to set the catalyst trim effectiveness value of the multi-component catalyst composition to the initial value, the at least one processor is configured to execute the stored instruction to perform actions comprising: calculating the initial value based on catalyst activation fundamentals including one or more of: an amount of activator in the multi-component catalyst composition, an ambient temperature, a contact time, and a trim solvent.

Statement 3. The soft sensor system of statement 1, wherein, to set the catalyst trim effectiveness value of the multi-component catalyst composition to the initial value, the at least one processor is configured to execute the stored instruction to perform actions comprising: setting the initial value to a predetermined initial value.

Statement 4. The soft sensor system of any of statements 1-3, wherein the properties of the PE resins of the polymerization process dataset include one or more of: density, melt index (MI), and melt index ratio (MIR) of each of the PE resins, and wherein the set of trained process models comprise a density process model, a MI process model, and a MIR process model.

Statement 5. The soft sensor system of any of statements 1-4, wherein each respective trained process model of the set of one or more trained process models is configured to predict a corresponding one of the properties of the PE resins based on an activated form of the multi-component catalyst composition, a temperature of a fluidized bed reactor of the gas phase reactor system, a ratio of hydrogen gas to alpha-olefin monomer in the fluidized bed reactor of the gas phase reactor system, a ratio of alpha-olefin comonomer to the alpha-olefin monomer in the fluidized bed reactor of the gas phase reactor system, an amount of induced condensing agent (ICA) in the fluidized bed reactor of the gas phase reactor system, a partial pressure of the alpha-olefin monomer in the fluidized bed reactor of the gas phase reactor system, and a residence time of the PE resins in the fluidized bed reactor of the gas phase reactor system.

Statement 6. The soft sensor system of statement 5, wherein the multi-component catalyst composition is a dual-component catalyst having a first catalyst compound and a second catalyst compound loaded onto a support, wherein the second catalyst compound is also a trim catalyst, and wherein the activated form of the multi-component catalyst composition is calculated as an amount of the first catalyst loaded onto the support divided by a sum of the amount of the first catalyst compound loaded onto the support, an amount of the second catalyst compound loaded onto the support, and the multiplication product of an amount of the second catalyst compound provided as trim catalyst and the catalyst trim effectiveness value.

Statement 7. The soft sensor system of any of statements 1-6, wherein the at least one processor is configured to execute the stored instruction to perform actions comprising: (F) receiving a set of operating parameters for the gas phase reactor system; and (G) using the received set of operating parameters, the catalyst trim effectiveness value, and at least one trained process model of the set of trained process models to determine at least one predicted property of a potential PE resin that would be produced using the multi-component catalyst composition and the gas phase reactor system according to the received set of operating parameters (H) controlling the gas phase reactor system to produce a PE resin product based at least in part upon the at least one predicted property of the potential PE resin; wherein controlling the gas phase reactor system comprises either adjusting one or more operating parameters of the gas phase reactor system, or maintaining constant the one or more operating parameters of the gas phase reactor system; and further wherein a PE resin product is produced based upon the (H) controlling.

Statement 8. The soft sensor system of any of statements 1-6, wherein the at least one processor is configured to execute the stored instruction to perform actions comprising (F) receiving a set of desired properties for a potential PE resin; and (G) using the received set of desired properties, the catalyst trim effectiveness value, and the set of trained process models to determine one or more predicted operating parameters of the gas phase reactor system to be used to produce the potential PE resin having the received set of desired properties using the multi-component catalyst composition; further wherein the potential PE resin is produced using the one or more predicted operating parameters of the gas phase reactor system.

Statement 9. The soft sensor system of any of statements 1-8, wherein the multi-component catalyst composition comprises a support comprising silica, an activator comprising an aluminoxane, and two metallocene catalysts.

Statement 10. The soft sensor system of statement 9, wherein the two metallocene catalysts comprise a bridged bis-cyclopentadienyl hafnocene and an unbridged indenyl-cyclopentadienyl zirconocene.

Statement 11. A method comprising: (A) obtaining a polymerization process dataset for a multi-component catalyst composition, wherein the polymerization process dataset includes one or more properties of polyethylene (PE) resins polymerized from ethylene monomers and alpha-olefin comonomers using the multi-component catalyst composition and different operating parameters of a gas phase reactor system; (B) setting a catalyst trim effectiveness value of the multi-component catalyst composition to an initial value; (C) training a respective process model for each of the one or more properties of the PE resins of the polymerization process dataset using the catalyst trim effectiveness value of the multi-component catalyst composition to yield a set of one or more trained process models; (D) optimizing the catalyst trim effectiveness value of the multi-component catalyst composition to minimize a sum of squared error (SSE) value of model residuals based on the set of one or more trained process models; (E) repeating steps (C) and (D) until a decrease in the minimized SSE value is less than a predefined threshold value; and (F) outputting or storing the optimized catalyst trim effectiveness value and the set of one or more trained process models for the multi-component catalyst composition.

Statement 12. The method of statement 11, wherein the one or more properties of the PE resins of the polymerization process dataset include one or more of: density, melt index (MI), and melt index ratio (MIR) of each of the PE resins, and wherein the set of trained process models comprise a density process model, a MI process model, and a MIR process model.

Statement 13. The method of any of statements 11 and 12, wherein each respective trained process model of the set of one or more trained process models is configured to predict a corresponding one of the properties of the PE resins based on an activated form of the multi-component catalyst composition, a temperature of a fluidized bed reactor of the gas phase reactor system, a ratio of hydrogen gas to alpha-olefin monomer in the fluidized bed reactor of the gas phase reactor system, a ratio of alpha-olefin comonomer to the alpha-olefin monomer the fluidized bed reactor of the gas phase reactor system, an amount of induced condensing agent (ICA) in the fluidized bed reactor of the gas phase reactor system, a partial pressure of the alpha-olefin monomer in the fluidized bed reactor of the gas phase reactor system, and a residence time of the PE resins in the fluidized bed reactor of the gas phase reactor system.

Statement 14. The method of any of statements 11-13, further comprising: (F) receiving a set of operating parameters for the gas phase reactor system; (G) using the received set of operating parameters, the catalyst trim effectiveness value, and at least one trained process model of the set of trained process models to determine at least one predicted property of a potential PE resin that would be produced using the multi-component catalyst composition and the gas phase reactor system according to the received set of operating parameters; (H) controlling the gas phase reactor system to produce a PE resin product based at least in part upon the at least one predicted property of the potential PE resin; wherein controlling the gas phase reactor system comprises either adjusting one or more operating parameters of the gas phase reactor system, or maintaining constant the one or more operating parameters of the gas phase reactor system; and (G) producing the PE resin product.

Statement 15. The method of any of statements 11-13, further comprising: (F) receiving a set of desired properties for a potential PE resin; (G) using the received set of desired properties, the catalyst trim effectiveness value, and the set of trained process models to determine one or more predicted operating parameters of the gas phase reactor system to be used to produce the potential PE resin having the received set of desired properties using the multi-component catalyst composition; and (H) producing the potential PE resin using the one or more predicted operating parameters of the gas phase reactor system.

Statement 16. The method of any statements 11-14, wherein the alpha-olefin comonomer comprises one or more C2 to C20 alpha-olefin comonomers.

Statement 17. The method of statement 16, wherein the alpha-olefin comonomer comprises one or more C3 to C12 alpha-olefin comonomers.

Statement 18. The method of statement 17, wherein the alpha-olefin comonomer comprises 1-butene, 1-hexene, 1-octene, or combinations thereof.

Statement 19. A non-transitory, computer-readable medium storing instructions executable by a processor of a soft sensor system, the instructions comprising instructions to: (A) receive a polymerization process dataset for a multi-component catalyst composition, wherein the polymerization process dataset includes one or more properties of polyethylene (PE) resins polymerized from ethylene monomers and alpha-olefin comonomers using the multi-component catalyst composition and different operating parameters of a gas phase reactor system; (B) set a catalyst trim effectiveness value of the multi-component catalyst composition to an initial value; (C) train a respective process model for each of the one or more properties of the PE resins of the polymerization process dataset using the catalyst trim effectiveness value of the multi-component catalyst composition to yield a set of one or more trained process models; (D) optimize the catalyst trim effectiveness value of the multi-component catalyst composition to minimize a sum of squared error (SSE) value of model residuals based on the set of trained process models; (E) repeat steps (C) and (D) until a decrease in the minimized SSE value is less than a predefined threshold value; and (F) output or store the optimized catalyst trim effectiveness value and the set of one or more trained process models for the multi-component catalyst composition.

Statement 20. The medium of statement 19, wherein the soft sensor system is communicatively coupled to one or more components of the gas phase reactor system, and wherein the instructions further comprise instructions to: (F) receive a set of operating parameters for the gas phase reactor system from the one or more components of the gas phase reactor system; (G) use the received set of operating parameters, the catalyst trim effectiveness value, and at least one of trained process model of the set of trained process models to determine at least one predicted property of a potential PE resin that would be produced using the multi-component catalyst composition and the gas phase reactor system according to the received set of operating parameters; and (H) control the gas phase reactor system to produce a PE resin product based at least in part upon the at least one predicted property of the potential PE resin; wherein controlling the gas phase reactor system comprises either adjusting one or more operating parameters of the gas phase reactor system, or maintaining constant the one or more operating parameters of the gas phase reactor system; and further wherein a PE resin product is produced based upon the (H) controlling.

Statement 21. The medium of statement 19, wherein the soft sensor system is communicatively coupled to one or more components of the gas phase reactor system, and wherein the instructions further comprise instructions to: (F) receive a set of desired properties for a potential PE resin; (G) use the received set of desired properties, the catalyst trim effectiveness value, and the set of trained process models to determine one or more predicted, operating parameters of the gas phase reactor system to be used to produce the potential PE resin having the received set of desired properties using the multi-component catalyst composition; and (H) adjust a configuration of at least one component of the gas phase reactor system based on the one or more predicted operating parameters of the gas phase reactor system to produce the potential PE resin; further wherein the potential PE resin is produced using the one or more predicted operating parameters of the gas phase reactor system.

While the disclosure has been described with respect to a number of embodiments and examples, those skilled in the art, having benefit of this disclosure, will appreciate that other embodiments can be devised which do not depart from the scope and spirit of the disclosure as disclosed herein. Although individual embodiments are discussed, the present disclosure covers all combinations of all those embodiments.

While compositions, methods, and processes are described herein in terms of “comprising,” “containing,” “having,” or “including” various components or steps, the compositions and methods can also “consist essentially of” or “consist of” the various components and steps. The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the disclosure, additionally, they do not exclude impurities and variances normally associated with the elements and materials used.

All numerical values within the detailed description are modified by “about” the indicated value, and take into account experimental error and variations that would be expected by a person having ordinary skill in the art.

Many alterations, modifications, and variations will be apparent to those skilled in the art in light of the foregoing description without departing from the spirit or scope of the present disclosure and that when numerical lower limits and numerical upper limits are listed herein, ranges from any lower limit to any upper limit are contemplated.

1. A soft sensor system, comprising:
at least one memory configured to store a polymerization process dataset for a multi-component catalyst composition, wherein the polymerization process dataset includes one or more properties of polyethylene (PE) resins prepared using the multi-component catalyst composition and different operating parameters of a gas phase reactor system; and
at least one processor configured to execute stored instruction to perform actions comprising:
(A) setting a catalyst trim effectiveness value of the multi-component catalyst composition to an initial value;
(B) training a respective process model for each of the one or more properties of the PE resins of the polymerization process dataset using the catalyst trim effectiveness value of the multi-component catalyst composition to yield a set of one or more trained process models;
(C) optimizing the catalyst trim effectiveness value of the multi-component catalyst composition to minimize a sum of squared error (SSE) value of model residuals based on the set of trained process models;
(D) repeating steps (B) and (C) until a decrease in the minimized SSE value is less than a predefined threshold value; and
(E) outputting or storing the optimized catalyst trim effectiveness value and the set of one or more trained process models for the multi-component catalyst composition.

2. The soft sensor system of claim 1, wherein, to set the catalyst trim effectiveness value of the multi-component catalyst composition to the initial value, the at least one processor is configured to execute the stored instruction to perform actions comprising:
calculating the initial value based on catalyst activation fundamentals including one or more of: an amount of activator in the multi-component catalyst composition, an ambient temperature, a contact time, and a trim solvent.

3. The soft sensor system of claim 1, wherein, to set the catalyst trim effectiveness value of the multi-component catalyst composition to the initial value, the at least one processor is configured to execute the stored instruction to perform actions comprising:
setting the initial value to a predetermined initial value.

4. The soft sensor system of claim 1, wherein the properties of the PE resins of the polymerization process dataset include one or more of: density, melt index (MI), and melt index ratio (MIR) of each of the PE resins, and wherein the set of trained process models comprise a density process model, a MI process model, and a MIR process model.

5. The soft sensor system of claim 1, wherein each respective trained process model of the set of one or more trained process models is configured to predict a corresponding one of the properties of the PE resins based on (i) an activated form of the multi-component catalyst composition, (ii) a temperature of a fluidized bed reactor of the gas phase reactor system, (iii) a ratio of hydrogen gas to alpha-olefin monomer in the fluidized bed reactor of the gas phase reactor system, (iv) a ratio of alpha-olefin comonomer to the alpha-olefin monomer in the fluidized bed reactor of the gas phase reactor system, (v) an amount of induced condensing agent (ICA) in the fluidized bed reactor of the gas phase reactor system, (vi) a partial pressure of the alpha-olefin monomer in the fluidized bed reactor of the gas phase reactor system, and (vii) a residence time of the PE resins in the fluidized bed reactor of the gas phase reactor system.

6. The soft sensor system of claim 5, wherein the multi-component catalyst composition is a dual-component catalyst having a first catalyst compound and a second catalyst compound loaded onto a support, wherein the second catalyst compound is also a trim catalyst, and wherein the activated form of the multi-component catalyst composition is calculated as an amount of the first catalyst compound loaded onto the support divided by a sum of (i) the amount of the first catalyst compound loaded onto the support, (ii) an amount of the second catalyst compound loaded onto the support, and (iii) the multiplication product of an amount of the second catalyst compound provided as trim catalyst and the catalyst trim effectiveness value.

7. The soft sensor system of claim 1, wherein the at least one processor is configured to execute the stored instruction to perform actions comprising:
(F) receiving a set of operating parameters for the gas phase reactor system; and
(G) using the received set of operating parameters, the catalyst trim effectiveness value, and at least one trained process model of the set of trained process models to determine at least one predicted property of a potential PE resin that would be produced using the multi-component catalyst composition and the gas phase reactor system according to the received set of operating parameters;
(H) controlling the gas phase reactor system to produce a PE resin product based at least in part upon the at least one predicted property of the potential PE resin;
wherein controlling the gas phase reactor system comprises either adjusting one or more operating parameters of the gas phase reactor system, or maintaining constant the one or more operating parameters of the gas phase reactor system; and
further wherein a PE resin product is produced based upon the (H) controlling.

8. The soft sensor system of claim 1, wherein the at least one processor is configured to execute the stored instruction to perform actions comprising:
(F) receiving a set of desired properties for a potential PE resin; and
(G) using the received set of desired properties, the catalyst trim effectiveness value, and the set of trained process models to determine one or more predicted operating parameters of the gas phase reactor system to be used to produce the potential PE resin having the received set of desired properties using the multi-component catalyst composition;
further wherein the potential PE resin is produced using the one or more predicted operating parameters of the gas phase reactor system.

9. The soft sensor system of claim 1, wherein the multi-component catalyst composition comprises a support comprising silica, an activator comprising an aluminoxane, and two metallocene catalysts.

10. The soft sensor system of claim 9, wherein the two metallocene catalysts comprise a bridged bis-cyclopentadienyl hafnocene and an unbridged indenyl-cyclopentadienyl zirconocene.

11. A method comprising:
(A) obtaining a polymerization process dataset for a multi-component catalyst composition, wherein the polymerization process dataset includes one or more properties of polyethylene (PE) resins polymerized from ethylene monomers and alpha-olefin comonomers using the multi-component catalyst composition and different operating parameters of a gas phase reactor system;
(B) setting a catalyst trim effectiveness value of the multi-component catalyst composition to an initial value;
(C) training a respective process model for each of the one or more properties of the PE resins of the polymerization process dataset using the catalyst trim effectiveness value of the multi-component catalyst composition to yield a set of one or more trained process models;
(D) optimizing the catalyst trim effectiveness value of the multi-component catalyst composition to minimize a sum of squared error (SSE) value of model residuals based on the set of one or more trained process models;
(E) repeating steps (C) and (D) until a decrease in the minimized SSE value is less than a predefined threshold value; and
(F) outputting or storing the optimized catalyst trim effectiveness value and the set of one or more trained process models for the multi-component catalyst composition.

12. The method of claim 11, wherein the one or more properties of the PE resins of the polymerization process dataset include one or more of: density, melt index (MI), and melt index ratio (MIR) of each of the PE resins, and wherein the set of trained process models comprise a density process model, a MI process model, and a MIR process model.

13. The method of claim 11, wherein each respective trained process model of the set of one or more trained process models is configured to predict a corresponding one of the properties of the PE resins based on: (i) an activated form of the multi-component catalyst composition, (ii) a temperature of a fluidized bed reactor of the gas phase reactor system, (iii) a ratio of hydrogen gas to alpha-olefin monomer in the fluidized bed reactor of the gas phase reactor system, (iv) a ratio of alpha-olefin comonomer to the alpha-olefin monomer in the fluidized bed reactor of the gas phase reactor system, (v) an amount of induced condensing agent (ICA) in the fluidized bed reactor of the gas phase reactor system, (vi) a partial pressure of the alpha-olefin monomer in the fluidized bed reactor of the gas phase reactor system, and (vii) a residence time of the PE resins in the fluidized bed reactor of the gas phase reactor system.

14. The method of claim 11, further comprising:
(F) receiving a set of operating parameters for the gas phase reactor system;
(G) using the received set of operating parameters, the catalyst trim effectiveness value, and at least one trained process model of the set of trained process models to determine at least one predicted property of a potential PE resin that would be produced using the multi-component catalyst composition and the gas phase reactor system according to the received set of operating parameters;
(H) controlling the gas phase reactor system to produce a PE resin product based at least in part upon the at least one predicted property of the potential PE resin;
wherein controlling the gas phase reactor system comprises either adjusting one or more operating parameters of the gas phase reactor system, or maintaining constant the one or more operating parameters of the gas phase reactor system; and
(G) producing the PE resin product.

15. The method of claim 11, further comprising:
(F) receiving a set of desired properties for a potential PE resin;
(G) using the received set of desired properties, the catalyst trim effectiveness value, and the set of trained process models to determine one or more predicted operating parameters of the gas phase reactor system to be used to produce the potential PE resin having the received set of desired properties using the multi-component catalyst composition; and
(H) producing the potential PE resin using the one or more predicted operating parameters of the gas phase reactor system.

16. The method of claim 11, wherein the alpha-olefin comonomer comprises one or more C₃ to C₂₀ alpha-olefin comonomers.

17. The method of claim 16, wherein the alpha-olefin comonomer comprises 1-butene, 1-hexene, 1-octene, or combinations thereof.

18. A non-transitory, computer-readable medium storing instructions executable by a processor of a soft sensor system, the instructions comprising instructions to:
(A) receive a polymerization process dataset for a multi-component catalyst composition, wherein the polymerization process dataset includes one or more properties of polyethylene (PE) resins polymerized from ethylene monomers and alpha-olefin comonomers using the multi-component catalyst composition and different operating parameters of a gas phase reactor system;
(B) set a catalyst trim effectiveness value of the multi-component catalyst composition to an initial value;
(C) train a respective process model for each of the one or more properties of the PE resins of the polymerization process dataset using the catalyst trim effectiveness value of the multi-component catalyst composition to yield a set of one or more trained process models;
(D) optimize the catalyst trim effectiveness value of the multi-component catalyst composition to minimize a sum of squared error (SSE) value of model residuals based on the set of trained process models;
(E) repeat steps (C) and (D) until a decrease in the minimized SSE value is less than a predefined threshold value; and
(F) output or store the optimized catalyst trim effectiveness value and the set of one or more trained process models for the multi-component catalyst composition.

19. The medium of claim 18, wherein the soft sensor system is communicatively coupled to one or more components of the gas phase reactor system, and wherein the instructions further comprise instructions to:
(F) receive a set of operating parameters for the gas phase reactor system from the one or more components of the gas phase reactor system;
(G) use the received set of operating parameters, the catalyst trim effectiveness value, and at least one of trained process model of the set of trained process models to determine at least one predicted property of a potential PE resin that would be produced using the multi-component catalyst composition and the gas phase reactor system according to the received set of operating parameters; and
(H) control the gas phase reactor system to produce a PE resin product based at least in part upon the at least one predicted property of the potential PE resin;
wherein controlling the gas phase reactor system comprises either adjusting one or more operating parameters of the gas phase reactor system, or maintaining constant the one or more operating parameters of the gas phase reactor system; and
further wherein a PE resin product is produced based upon the (H) controlling.

20. The medium of claim 18, wherein the soft sensor system is communicatively coupled to one or more components of the gas phase reactor system, and wherein the instructions further comprise instructions to:
(F) receive a set of desired properties for a potential PE resin;
(G) use the received set of desired properties, the catalyst trim effectiveness value, and the set of trained process models to determine one or more predicted operating parameters of the gas phase reactor system to be used to produce the potential PE resin having the received set of desired properties using the multi-component catalyst composition; and
(H) adjust a configuration of at least one component of the gas phase reactor system based on the one or more predicted operating parameters of the gas phase reactor system to produce the potential PE resin;
further wherein the potential PE resin is produced using the one or more predicted operating parameters of the gas phase reactor system.
